(19) United States
(12) Patent Application Publication
(10) Pub. No.: US 2007/0037154 A1
(43) Pub. Date: Feb. 15, 2007

(54) Fine mapping of chromosome 17 quantitative trait loci and use of same for marker assisted selection

(75) Inventors: Max F. Rothschild, Ames, IA (US); Antonio Marcos Costa Do Amaral Ramos, Ames, IA (US); Kwan-Suk Kim, Cheong-ju (KR)

Correspondence Address: MCKEE, VOORHEES & SEASE, P.L.C., 801 GRAND AVENUE, SUITE 3200, DES MOINES, IA, 50309-2721, US

(73) Assignee: IOWA STATE UNIVERSITY RESEARCH FOUNDATION INC., AMES, IA (US)

(21) Appl. No.: 10/555,809

(22) PCT Filed: May 24, 2004
(86) PCT No.: PCT/US04/16418

§ 371 Date: Jul. 20, 2006

Related U.S. Application Data
(60) Provisional application No. 60/473,179, filed on May 23, 2003.

(51) Int. Cl. C12Q 1/68

(52) U.S. Cl. 435/6

(57) ABSTRACT

Disclosed herein is fine mapping of a quantitative trait locus on Chromosome 17 which is associated with meat traits, growth and fatness. The quantitative trait locus correlates with several major effect genes which have phenotypic correlations with animal growth and meat quality which may be used for marker assisted breeding. Specific polymorphic alleles of these genes are disclosed for tests to screen animals to determine those more likely to produce desired traits.

### CROSS-REFERENCE TO RELATED APPLICATIONS

This application claims priority to U.S. Provisional Application Ser. No. 60/473,179, filed May 23, 2003, which is herein incorporated by reference in its entirety.

### GRANT REFERENCE

Work for this invention was funded in part by USDA/CSREES Contract No's. 2003- 31100-06019 and 2002-31100-06019. The Government may have certain rights in this invention.

### FIELD OF THE INVENTION

This invention relates generally to the detection of genetic differences among animals. More particularly, the invention relates to genetic variation that is indicative of heritable phenotypes associated with higher meat quality and growth rate and fat deposition. Methods and compositions for use of specific genetic markers and chromosomal regions associated with the variation in genotyping of animals and selection are also disclosed.

### BACKGROUND OF THE INVENTION

Researchers have found that quantitative trait phenotypes are continuously distributed in natural populations, due to segregation of alleles at multiple genes in different regions. These quantitative trait loci (QTL) combined with differences in environmental sensitivity of QTL alleles affect the phenotypes. Determining the genetic and environmental bases of variation for quantitative traits is important for human health, agriculture, and the study of evolution. But, complete genetic dissection of quantitative traits is currently feasible only in genetically tractable and well characterized model systems. (Mackay, Nat. Rev. Genet. 2:11-20 (2001); Wright et al., Genome Biol. 2: 2007.1-2007.8 (2001)). For example, the number of genes involved in quantitative genetic variation is not known, the number and effects of individual alleles at these genes, or the gene action is also generally unknown. To date, genes and causal variants have been detected for very few quantitative traits. For example, such quantitative traits such as double-muscling in cattle (Grobet et al., Mamm. Genome 9:210-213 (1998), alteration in fruit size (Frary et al., Science 289:85-88 (2000), growth and performance traits in pigs (Kim et al., Mamm. Genome 11:131-135 (2000), excess glycogen content in pig skeletal muscle (Milan et al., Science 288:1248-1251 (2000), and increased ovulation and litter size in sheep (Wilson et al., Biol Reprod. 64:1225-1235 (2001). The effects of the mutations in the majority of these examples are so large that the phenotypes segregate almost as Mendelian traits.

To understand and exploit the genetics of complex quantitative traits, experimental populations derived from two lines differing widely for traits of interest have been successfully used in model species (Belknap et al., Behav. Genet. 23:213-222 (1993); Talbot et al., Nat. Genet. 21:305-308 (1999)), plants (Paterson et al., Nature 335:721-726 (1988)), and livestock (Andersson et al., Science 263:1771-1774 (1994)) to detect quantitative trait loci (QTL). These studies have succeeded in mapping QTL for which alleles differ in frequency between the parental populations, for example, between commercial agricultural cultivars and wild-type populations (Paterson et al., Nature 335:721-726 (1988); Andersson et al., Science 263:1771-1774 (1994)). In addition to understanding the architecture of quantitative traits, crosses involving agricultural species are also motivated by the potential to exploit variation within elite populations; commercial plant and animal populations are usually not based upon the same crosses that are used in the QTL detection studies but the power of linkage studies in line crosses is generally greater than that of studies within populations. In commercial pig breeding populations, for example, elite populations comprise closed outbred populations that have been subjected to selection over a number of generations to improve their commercial performance, whereas wild boar (Andersson et al., Science 263:1771-1774 (1994)) and Chinese Meishan (Walling et al. Anim. Genet; 29:415-424 (1998); De Koning et al, Genetics 152:1679-1690 (1999); De Koning et al, Proc. Natl. Acad. Sci. USA 97:7947-7950 (2000); Bidanel et al., Genet. Sel. Evol. 33:289-309 (2001)) populations have been often employed in QTL studies. The implicit hypothesis in many QTL studies using divergent lines is that knowledge of between-population genetic variation can be extrapolated to genetic variation in other populations or species. Segregation at QTL in commercial populations can be utilized by breeders through gene- or marker-assisted selection programs (e.g., Dekkers and Hospital, Nat. Rev. Genet. 3:22-32 (2002)).

Selection for meat and fat production, for example, in pigs has taken place for centuries, but intense selection using modern statistical methods has been practiced for only the past ˜50 years (Clutter, A. C., and E. W. Brascamp, 1998 Genetics of performance traits, pp. 427-462 in The Genetics of the Pig, edited by M. F. Rothschild and A. Ruvinsky. CAB International, Wallingford, UK).

Until recently, it has been impracticable to identify the genes that are responsible for variation in continuous traits, or to directly observe the effects of their different alleles. But now, the abundance of genetic markers has made it possible to identify quantitative trait loci (QTL)—the regions of a chromosome or, individual sequence variants that are responsible for trait variation. (Barton et al., Nat. Rev. Genet 3:11-21 (2002)). To the extent that genes are conserved among species and animals, it is expected that the different alleles will also correlate with variability in certain gene(s) as well as in economic or meat-producing animal species such as cattle, sheep, chicken, etc. There are instances of conserved polymorphisms among species. For example, Nonneman et al. recently discovered a polymorphism in exon 2 of the porcine TBG gene that results in the amino acid change of the consensus histidine to an asparagine. This SNP resides in the ligand-binding domain of the mature polypeptide and the Meishan allele is the conserved allele found in human, bovine, sheep and rodent TBG. Mutations in this region of human TBG result in decreased heat stability and affinity for ligand. Functional studies indicate altered binding characteristics of the TBG isoforms. Nonneman et al., Plant & Animal Genomes XII Conference, “Functional Validation of A Polymorphism for Testis Size on the Porcine X Chromosome”, Jan. 10-14, 2004, Town & Country Convention Center, San Diego, Calif. Additionally, Winter et al. finds that increased milk fat content in different breeds is strongly associated with a lysine at position 232 of the protein encoded by bovine DGAT. An alignment of DGAT1 amino acid sequences of different plant and animal species indicates a conserved lysine residue at position 232 of the bovine sequence. Winter et al., Proc Natl Acad Sci USA. Jul. 9; 99(14:9300-9305 (2002). Furthermore, a conserved mutation in the MATP gene has been identified, which causes the cream coat color in the horse. This conserved mutation was also described in mice and humans, but not in medaka Mariat et al., Genet Sel Evol. Jan-Feb; 35(1): 119-33 (2003).

There have also been instances of conservation of a gene across species. Many genes involved in fundamental biological processes have been conserved as species have evolved, i.e., many genes are similar in different species. The MC1-R gene has been indicated to be a well-conserved gene having no other fundamental function beside pigmentation. In several species, mutations in the MC1-R gene have been shown to cause the dominant expression of black pigment. Klungland et al., Pigmentary Switches in Domestic Animal Species Annals of the New York Academy of Sciences, 994:331-338 (2003). A specific protein-DNA interaction was found to be blocked by a single base pair change in the binding site of glucocorticoid receptor protein (GCR). Moreover it is reported that all three putative domains (the steroid binding, immunoreactive, and DNA binding) have been conserved between two divergent species, pig and rat. Marks et al., J Steroid Biochem. Jun; 24(6): 1097-103 (1986).

An example of a conserved gene order is demonstrated by Seroude et al. (Mammalian Genomics, Jun; 10(6) 565-8 (1999)) wherein a radiation hybrid map of the Chromosome 15 q2.3-q2.6 region containing the RN gene was constructed, which has large effects on glycogen content in muscle and meat quality. Ten microsatellites and eight genes were mapped. They found that the relative order of genes AE3 and INHA was inverted on the porcine physical map in comparison with the mouse linkage map, but the order of other genes already mapped in the mouse was identical to pigs. Moreover, they found no clear difference between the gene order in pig Chromosome 15 and human Chromosome 2q. Based on the evolutionary link and comparative genomics of animals, it can be determined whether the variation in a gene is or is likely to be associated with a functional trait between closely linked species.

Indeed, the best approach to genetically improve economic traits is to find relevant chromosomal regions and then genetic -markers directly in the population under selection. Phenotypic measurements can be performed continuously on some animals from the nucleus population of breeding organizations. This phenotypic data is collected in order to enable the detection of relevant genetic markers, and to validate markers identified using experimental populations or to test candidate genes.

Not all genes have an easily identifiable common functional variant that can be exploited in association studies, and in many gene cases researchers have identified only changes in individual nucleotides (i.e., single nucleotide polymorphisms (SNPs)) that have no known functional significance. Nevertheless, SNPs are potentially useful in narrowing a linkage region with in a chromosome. In addition, SNPs may show a statistically significant association with a quantitative trait if located within or near that gene by virtue of linkage disequilibrium.

Significant markers or genes can then be included directly in the selection process. An advantage of the molecular information is that we can obtain it already at very young age of the breeding animal, which means that animals can be preselected based on DNA markers before the growing performance test is completed. This is a great advantage for the overall testing and selection system.

Polymorphisms hold promise for use as genetic markers in determining which genes contribute to multigenic or quantitative traits, suitable markers and suitable methods for exploiting those markers are beginning to be brought to bear on the genes related to growth and meat quality.

It can be seen from the foregoing that a need exists for identification of genetic variation associated with or in linkage disequilibrium with, genomic regions, which may be used to improve economically beneficial characteristics in animals by identifying and selecting animals with the improved characteristics at the genetic level.

Another object of the invention is to identify a genetic locus in which the variation present has a quantitative effect on a phenotypic trait of interest to breeders.

Another object of the invention is to provide a specific assay for determining the presence of such genetic variation.

A further object of the invention is to provide a method of evaluating animals that increases accuracy of selection and breeding methods for desired traits.

Yet another object of the invention is to provide a PCR amplification test to greatly expedite the determination of presence of the marker(s) of such quantitative trait variation.

Additional objects and advantages of the invention will be set forth in part in the description that follows, and in part will be obvious from the description, or may be learned by the practice of the invention. The objects and advantages of the invention will be attained by means of the instrumentalities and combinations particularly pointed out in the appended claims.

### BRIEF SUMMARY OF THE INVENTION

The methods of the present invention comprise the use of nucleic acid markers genetically linked to loci associated with economically important traits. The markers are used in genetic mapping of genetic material of animals to be used in and/or which have been developed in a breeding program, allowing for marker-assisted selection to identify or to move traits into elite germplasm. The invention relates to the discovery of genetic variation in genomic regions associated with or in linkage disequilibrium or otherwise genetically linked therewith that may be used to predict phenotypic traits in animals. According to an embodiment of the invention, specific regions of chromosome 17 have been fine mapped and shown to be quantitative trait loci for various traits. Namely the region of chromosome 17 at 70 to 108cM have been identified as quantitative trait loci for growth traits. More specific regions within this area have been identified for meat quality and fatness. Further several genes located in this region have been shown to be polymorphic and thus useful as genetic markers for these QTL. This includes PKIG, MMP9, PTPN1, ATP9A, CYP24A1, DOK5, MC3R, AURKA, SPO11, RAE1, PCK1, RAB22A, GNAS, CTSZ, and PPP1R3D. To the extent that these genes are conserved among species and animals, and it is expected that the different alleles disclosed herein will also correlate with variability in these gene(s) in other economic or meat-producing animals such as cattle, sheep, chicken, etc.

An embodiment of the invention is a method of identifying an allele that is associated with meat quality traits comprising obtaining a tissue or body fluid sample from an animal; amplifying DNA present in said sample comprising a region 70 -107 cM of chromosome 17 linked to a nucleotide sequence which encodes PKIG, MMP9, PTPN1, ATP9A, CYP24A1, DOK5, MC3R, AURKA, SPO11, RAE1, PCK1, RAB22A, GNAS, CTSZ, and PPP1R3D; and detecting the presence of a polymorphic variant of said nucleotide sequences wherein said variant is associated with phenotypic variation in meat quality.

Another embodiment of the invention is a method of determining a genetic marker which may be used to identify and select animals based upon their meat quality or growth traits comprising obtaining a sample of tissue or body fluid from said animals, said sample comprising DNA; amplifying DNA present in said sample in the region of chromosome 17, said region comprising a nucleotide sequence which encodes upon expression PKIG, MMP9, PTPN1, ATP9A, CYP24A1, DOK5, MC3R, AURKA, SPO11, RAE1, PCK1, RAB22A, GNAS, CTSZ, and PPP1R3D present in said sample from a first animal; determining the presence of a polymorphic allele present in said sample by comparison of said sample with a reference sample or sequence; correlating variability for growth or meat quality in said animals with said polymorphic allele; so that said allele may be used as a genetic marker for the same in a given group, population, or species.

Yet anther embodiment of the invention is a method of identifying an animal for its propensity for growth or meat quality traits, said method comprising obtaining a nucleic acid sample from said animal, and determining the presence of an allele characterized by a polymorphism in a PKIG, MMP9, PTPN1, ATP9A, CYP24A1, DOK5, MC3R, AURKA, SPO11, RAE1, PCK1, RAB22A, GNAS, CTSZ, and PPP1R3D coding sequence present in said sample, or a polymorphism in linkage disequilibrium therewith, said genotype being one which is or has been shown to be significantly associated with a trait indicative of growth or meat quality.

Additional embodiments are set forth in the Detailed Description of the Invention and in the Examples.

### BRIEF DESCRIPTION OF THE DRAWINGS

FIG. 1 shows F-ratio curves for evidence of QTL associated with meat quality on SSC 17. The x-axis indicates the relative position on a linkage map. The y-axis represents the F-ratio. Arrows on the x-axis indicate the position where a marker was present. Shown are traits of interest: AVGP=Average Glycolytic Potential; AVLAC=Average Lactate; COLOR=color; LABLM=Lab Loin Minolta; LABLH=Lab Loin Hunter.

FIG. 2A depicts a PCR-RFLP of a 330 bp fragment of the porcine Cathepsin Z (CTSZ) gene showing the expected digestion pattern with the enzyme AlwNI.

FIG. 2B depicts a PCR-RFLP of a 321 bp fragment of the porcine GNAS gene showing the expected digestion pattern with the enzyme BbsI.

FIG. 2C depicts a PCR-RFLP of the porcine MC3R gene showing the expected digestion pattern with the enzyme MnlI.

FIG. 3 shows the consensus sequence of CTSZ in pig.

FIG. 4 shows the consensus sequence of GNAS in pig.

FIG. 5 shows the consensus sequence of MC3R in pig.

FIG. 6 provides a map of genes mapped to chromosome 17.

FIG. 7 shows the consensus sequence of PKIG in pig. The position of a single nucleotide polymorphism is indicated with in bold.

FIG. 8 shows the consensus sequence of MMP9 in pig. The position of a single nucleotide polymorphism is indicated with in bold.

FIG. 9 shows the consensus sequence of PTPN1 in pig. The position of a single nucleotide polymorphism is indicated with in bold.

FIG. 10 shows the consensus sequence of ATP9A in pig. The position of a single nucleotide polymorphism is indicated with in bold.

FIG. 11 shows the consensus sequence of CYP24A1 in pig. The position of a single nucleotide polymorphism is indicated with in bold.

FIG. 12 shows the consensus sequence of DOK5 in pig. The position of a single nucleotide polymorphism is indicated with in bold.

FIG. 13 shows the consensus sequence of AURKA in pig. The position of a single nucleotide polymorphism is indicated with in bold.

FIG. 14 shows the consensus sequence of SPO11 in pig. The position of a single nucleotide polymorphism is indicated with in bold.

FIG. 15 shows the consensus sequence of RAE1 in pig. The position of a single nucleotide polymorphism is indicated with in bold.

FIG. 16 shows the consensus sequence of PCK1 in pig. The position of a single nucleotide polymorphism is indicated with in bold.

FIG. 17 shows the consensus sequence of RAB22A in pig. The position of a single nucleotide polymorphism is indicated with in bold.

FIG. 18 shows the consensus sequence of PPP1R3D in pig. The position of a single nucleotide polymorphism is indicated with in bold.

FIG. 19 shows the fine mapping of the QTL at chromosome 17 according to the invention.

### DETAILED DESCRIPTION OF THE PREFERRED EMBODIMENT

Genetic markers closely linked to important genes may be used to indirectly select for favorable alleles more efficiently than direct phenotypic selection (Lande and Thompson 1990). Therefore, it is of particular importance, both to the animal breeder and to farmers who grow and sell animals as a cash crop, to identify, through genetic mapping, the quantitative trait loci (QTL) for various economically valuable traits such as growth, meat quality and fatness. Knowing the QTLs associated with these traits animal breeders will be better able to breed animals which possess genotypic and phenotypic characteristics. To achieve the objects and in accordance with the purpose of the invention, as embodied and broadly described herein, the present invention provides the discovery of alternate chromosomal regions and genotypes which provide a method for genetically typing animals and screening animals to determine those more likely to possess favorable growth and less fat deposition and meat quality traits or to select against animals which have alleles indicating less favorable growth, are fatter and poorer meat quality traits and/or feed efficiency traits. As described herein, the effect on meat quality may be demonstrated through the use of a particular identifier, such as pH or drip loss, but the invention is not so limited. As used herein the use of any particular indicia of the phenotypic traits of growth or meat quality shall be interpreted to include all indicia for which variability is associated with the disclosed allele with respect to meat quality or growth or fatness. As used herein a “favorable growth, fatness, or meat quality trait” means a significant improvement (increase or decrease) in one of any measurable indicia of growth, or meat quality above the mean of a given population, so that this information can be used in breeding to achieve a uniform population which is optimized for these traits. This may include an increase in some traits or a decrease in others depending on the desired characteristics. For a review of some example economic traits the following may be consulted: Sosnicki, A. A., E. R. Wilson, E. B. Sheiss, A. deVries, 1998 “Is there a cost effective way to produce high quality pork?”, Reciprocal Meat Conference Proceedings, Vol. 51.

Methods for assaying for these traits generally comprises the steps 1) obtaining a biological sample from an animal; and 2) analyzing the genomic DNA or protein obtained in 1) to determine which allele(s) is/are present. Haplotype data which allows for a series of linked polymorphisms to be combined in a selection or identification protocol to maximize the benefits of each of these markers may also be used and are contemplated by this invention.

Since several of the polymorphisms may involve changes in amino acid composition of the respective protein or will be indicative of the presence of this change, assay methods may even involve ascertaning the amino acid composition of the protein of the major effect genes of the invention. Methods for this type or purification and analysis typically involve isolation of the protein through means including fluorescence tagging with antibodies, separation and purification of the protein (i.e., through reverse phase HPLC system), and use of an automated protein sequencer to identify the amino acid sequence present. Protocols for this assay are standard and known in the art and are disclosed in Ausubel et al. (eds.), Short Protocols in Molecular Biology, Fourth ed. John Wiley and Sons 1999.

In another embodiment, the invention comprises a method for identifying genetic markers for growth, fatness and meat quality. Once a major effect gene has been identified, it is expected that other variation present in the same gene, allele or in sequences in useful linkage disequilibrium therewith may be used to identify similar effects on these traits without undue experimentation. The identification of other such genetic variation, once a major effect gene has been discovered, represents more than routine screening and optimization of parameters well known to those of skill in the art and is intended to be within the scope of this invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence. comparison; in this case, the Reference sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Optimal alignment of sequences for comparison may be conducted by the local homology algorithm of Smith and Waterman, Adv. Appl Math. 2:482 (1981); by the homology alignment algorithm of Needleman and Wunsch, J Mol Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al, Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff(1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (I) The term “substantial idenfity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g., the genes discussed herein), which have previously been shown to be associated with a particular trait. Thus, in the present situation, taking one or both of the genes, it would be possible, at least in the short term, to select for animals likely to produce desired traits, or alternatively against animals likely to produce less desirable traits indirectly, by selecting for certain alleles of an associated marker through the selection of specific alleles of alternative chromosome markers. As used herein the term “genetic marker” shall include not only the nucleotide polymorphisms disclosed by any means of assaying for the protein changes associated with the polymorphism, be they linked genetic markers in the same chromosomal region, use of microsatellites, or even other means of assaying for the causative protein changes indicated by the marker and the use of the same to influence traits of an animal.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

The invention is intended to include the disclosed sequences as well as all conservatively modified variants thereof. The terms PKIG, MMP9, PTPN1, ATP9A, CYP24A1, DOK5, MC3R, AURKA, SPO11, RAE1, PCK1, RAB22A, GNAS, CTSZ, and PPP1R3D as used herein shall be interpreted to include these conservatively modified variants. The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His).

Those of ordinary skill in the art will recognize that some substitution will not alter the activity of the polypeptide to an extent that the character or nature of the polypeptide is substantially altered. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with meat quality/growth-like characteristics. When it is desired to alter the amino acid sequence of a polypeptide to create an equivalent, or a variant or portion of a polypeptide of the invention, one skilled in the art will typically change one or more of the codons of the encoding DNA sequence according to Table 1 (See infra). For example, certain amino acids may be substituted for other amino acids in a protein structure without appreciable loss of activity. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences, which encode said peptides without appreciable loss of their biological utility or activity. A degenerate codon means that a different three letter codon is used to specify the same amino acid. For example, it is well known in the art that the following RNA codons (and therefore, the corresponding DNA codons, with a T substituted for a U) can be used interchangeably to code for each specific amino acid:

TABLE 1

| | |
|---|---|
| Amino Acids | Codons |

| | |
|---|---|
| Phenylalanine (Phe or F) | UUU, UUC, UUA or UUG |
| Leucine (Leu or L) | CUU, CUC, CUA or CUG |
| Isoleucine (Ile or I) | AUU, AUC or AUA |
| Methionine (Met or M) | AUG |
| Valine (Val or V) | GUU, GUC, GUA, GUG |
| Serine (Ser or S) | AGU or AGC |
| Proline (Pro or P) | CCU, CCC, CGA, CCG |
| Threonine (Thr or T) | ACU, ACC, ACA, ACG |
| Alanine (Ala or A) | GCU, GCG, GCA, GCC |
| Tryptophan (Trp) | UGG |
| Tyrosine (Tyr or Y) | UAU or UAC |
| Histidine (His or H) | CAU or CAC |
| Glutanilne (Gln or Q) | CAA or CAG |
| Asparagine (Asn or N) | AAU or AAG |
| Lysine (Lys or K) | AAA or AAG |
| Aspartic Acid (Asp or D) | GAU or GAC |
| Glutamic Acid (Glu or E) | GAA or GAC |
| Cysteine (Cys or C) | UGU or UGC |
| Arginine (Arg or R) | AGA or AGG |
| Glycine (Gly or G) | GGU or GGC or GGA or GGG |
| Termination codon | UAA, UAG or UGA |

An embodiment of the invention relates to genetic markers for economically valuable traits in animals. The markers represent polymorphic variation or alleles that are associated significantly with growth and/or meat quality and thus provide a method of screening animals to determine those more likely to produce desired traits. As used herein the term “marker” shall include a polymorphic variant capable of detection which may be linked to a quantitative trait loci and thus useful for assaying for the particular trait in the QTL.

Thus, the invention relates to genetic markers and methods of identifying those markers in an animal of a particular breed, strain, population, or group, whereby the animal is more likely to yield desired meat or growth or fatness traits.

Genetic Association with Meat Quality, Fatness and Growth Traits on Chromosome 17

Genetic analysis described herein led to the discovery of genetic association with meat quality, fatness and growth traits on chromosome 17. The association identifies chromosome 17 as the location of one or more chromosomal regions/DNA segments or genes associated with favorable meat quality, fatness, and growth traits in animals and of considerable effect size. In particular, chromosome 17 is identified as containing at least one DNA segment or gene associated with favorable meat quality, fatness and growth traits.

The finding of association of genetic markers/polymorphisms disclosed herein with meat quality, fatness and growth traits indicates that there is one or more meat quality and growth traits chromosomal regions/DNA segments or meat quality and growth traits genes on chromosome 17 that either directly cause or confer a significant improvement in one of any measurable indicia of growth, fatness or meat quality above the mean of a given population.

The discovery of one or more growth, fatness, or meat quality-associated genes on chromosome 17, as evidenced by significant association with growth, fatness, or meat quality on chromosome 17, thus provides the basis for genetic analysis methods described herein which include: methods of identifying an allele that is associated with meat quality, fatness, and growth traits; methods of determining a genetic marker which may be used and select animals based upon their meat quality or growth traits; methods of identifying an animal for its propensity for growth, fatness or meat quality traits.

Genetic Markers Associated with Growth, Fatness or Meat Quality Traits

Genetic markers associated with meat growth or meat quality traits are provided herein. The markers are located on porcine chromosome 17. In particular embodiments of the genetic markers found in PKIG, MMP9, PTPN1, ATP9A, CYP24A1, DOK5, MC3R, AURKA, SPO11, RAE1, PCK1, RAB22A, GNAS, CTSZ, and PPP1R3D were mapped underneath the SSC17 QTL peaks for traits disclosed herein. The markers can be identified through linkage disequilibrium or association assessment methods described herein or known to those of skill in the art, and provide scores or results indicative of linkage disequilibrium with a chromosomal region/DNA segment or gene or of association with growth, fatness or meat quality when tested by such assessment methods. The genetic markers are associated with growth or meat quality as individual markers and/or in combinations, such as haplotypes, that are associated with growth or meat quality.

Genetic Markers on Porcine Chromosome 17

A genetic marker is a DNA segment with an identifiable location in a chromosome. Genetic markers may be used in a variety of genetic studies such as, for example, locating the chromosomal position or locus of a DNA sequence of interest, and determining if a subject is predisposed to or has a particular trait.

Because DNA sequences that are relatively close together on a chromosome tend to be inherited together, tracking of a genetic marker through generations in a population and comparing its inheritance to the inheritance of another DNA sequence of interest can provide information useful in determining the relative position of the DNA sequence of interest on a chromosome. Genetic markers particularly useful in such genetic studies are polymorphic. Such markers also may have an adequate level of heterozygosity to allow a reasonable probability that a randomly selected animal will be heterozygous.

The occurrence of variant forms of a particular DNA sequence, e.g., a gene, is referred to as polymorphism. A region of a DNA segment in which variation occurs may be referred to as a polymorphic region or site. A polymorphic region can be a single nucleotide (single nucleotide polymorphism or SNP), the identity of which differs, e.g., in different alleles, or can be two or more nucleotides in length. For example, variant forms of a DNA sequence may differ by an insertion or deletion of one or more nucleotides, insertion of a sequence that was duplicated, inversion of a sequence or conversion of a single nucleotide to a different nucleotide. Each animal can carry two different forms of the specific sequence or two identical forms of the sequence.

Differences between polymorphic forms of a specific DNA sequence may be detected in a variety of ways. For example, if the polymorphism is such that it creates or deletes a restriction enzyme site, such differences may be traced by using restriction enzymes that recognize specific DNA sequences. Restriction enzymes cut (digest) DNA at sites in their specific recognized sequence, resulting in a collection of fragments of the DNA. When a change exists in a DNA sequence that alters a sequence recognized by a restriction enzyme to one not recognized the fragments of DNA produced by restriction enzyme digestion of the region will be of different sizes. The various possible fragment sizes from a given region therefore depend on the precise sequence of DNA in the region. Variation in the fragments produced is termed “restriction fragment length polymorphism” (RFLP). The different sized-fragments reflecting variant DNA sequences can be visualized by separating the digested DNA according to its size on an agarose gel and visualizing the individual fragments by annealing to a labeled, e.g., radioactively or otherwise labeled, DNA “probe”.

PCR-RFLP, broadly speaking, is a technique that involves obtaining the DNA to be studied, amplifying the DNA, digesting the DNA with restriction endonucleases, separating the resulting fragments, and detecting the fragments of various genes. The use of PCR-RFLPs is the preferred method of detecting the polymorphisms, disclosed herein. However, since the use of RFLP analysis depends ultimately on polymorphisms and DNA restriction sites along the nucleic acid molecule, other methods of detecting the polymorphism can also be used and are contemplated in this invention. Such methods include ones that analyze the polymorphic gene product and detect polymorphisms by detecting the resulting differences in the gene product.

SNP markers may also be used in fine mapping and association analysis, as well as linkage analysis (see, e.g., Kruglyak (1997) Nature Genetics 17:21-24). Although an SNP may have limited information content, combinations of SNPs (which individually occur about every 100-300 bases) may yield informative haplotypes. SNP databases are available. Assay systems for determining SNPs include synthetic nucleotide arrays to which labeled, amplified DNA is hybridized (see, e.g., Lipshutz et al. (1999) Nature Genet. 21:2-24); single base primer extension methods (Pastinen et al. (1997) Genome Res. 7:606-614), mass spectroscopy on tagged beads, and solution assays in which allele-specific oligonucleotides are cleaved orjoined at the position of the SNP allele, resulting in activation of a fluorescent reporter system (see, e.g., Landegren et al. (1998) Genome Res. 8:769-776).

Chromosome 17

Pig chromosome 17 is well conserved (homologous to human chromosome 20 and mouse chromosome 2).

Genetic Association

When two loci are extremely close together, recombination between them is very rare, and the rate at which the two neighboring loci recombine can be so slow as to be unobservable except over many generations. The resulting allelic association is generally referred to as linkage disequilibrium. Linkage disequilibrium can be defined as specific alleles at two or more loci that are observed together on a chromosome more often than expected from their frequencies in the population. As a consequence of linkage disequilibrium, the frequency of all other alleles present in a haplotype carrying a trait-causing allele will also be increased (just as the trait-causing allele is increased in an affected, or trait-positive, population) compared to the frequency in a trait-negative or random control population. Therefore, association between the trait and any allele in linkage disequilibrium with the trait-causing allele will suffice to suggest the presence of a trait-related DNA segment in that particular region of a chromosome. On this basis, association studies are used in methods of locating and discovering methods, as disclosed herein, of identifying an allele that is associated with meat quality and growth traits in animals.

A marker locus must be tightly linked to the trait locus in order for linkage disequilibrium to exist between the loci. In particular, loci must be very close in order to have appreciable linkage disequilibrium that may be useful for association studies. Association studies rely on the retention of adjacent DNA variants over many generations in historic ancestries, and, thus, trait-associated regions are theoretically small in outbred random mating populations.

The power of genetic association analysis to detect genetic contributions to traits can be much greater than that of linkage studies. Linkage analysis can be limited by a lack of power to exclude regions or to detect loci with modest effects. Association tests can be capable of detecting loci with smaller effects (Risch and Merikangas (1996) Science 273:1516-1517), which may not be detectable by linkage analysis.

The aim of association studies when used to discover genetic variation in genes associated with phenotypic traits is to identify particular genetic variants that correlate with the phenotype at the population level. Association at the population level may be used in the process of identifying a gene or DNA segment because it provides an indication that a particular marker is either a functional variant underlying the trait (i.e., a polymorphism that is directly involved in causing a particular trait) or is extremely close to the trait gene on a chromosome. When a marker analyzed for association with a phenotypic trait is a functional variant, association is the result of the direct effect of the genotype on the phenotypic outcome. When a marker being analyzed for association is an anonymous marker, the occurrence of association is the result of linkage disequilibrium between the marker and a functional variant.

There are a number of methods typically used in assessing genetic association as an indication of linkage disequilibrium, including case-control study of unrelated animals and methods using family-based controls. Although the case-control design is relatively simple, it is the most prone to identifying DNA variants that prove to be spuriously associated (i.e., association without linkage) with the trait. Spurious association can be due to the structure of the population studied rather than to linkage disequilibrium. Linkage analysis of such spuriously associated allelic variants, however, would not detect evidence of significant linkage because there would be no familial segregation of the variants. Therefore, putative association between a marker allele and a meat quality, fatness and growth trait identified in a case-control study should be tested for evidence of linkage between the marker and the disease before a conclusion of probable linkage disequilibrium is made. Association tests that avoid some of the problems of the standard case-control study utilize family-based controls in which parental alleles or haplotypes not transmitted to affected offspring are used as controls.

In contrast to genetic linkage, which is a property of loci, genetic association is a property of alleles. Association analysis involves a determination of a correlation between a single, specific allele and a trait across a population, not only within individual groups. Thus, a particular allele found through an association study to be in linkage disequilibrium with a meat quality or growth or fatness associated-allele can form the basis of a method of determining a predisposition to or the occurrence of the trait in any animal. Such methods would not involve a determination of phase of an allele and thus would not be limited in terms of the animals that may be screened in the method.

Methods for Identifying Genetic Markers Associated with Meat Quality, Growth or Fatness Traits

Also provided herein are methods of determining a genetic marker, which may be used to identify and select animals, based upon their meat quality or growth traits. The methods include a step of testing a polymorphic marker on chromosome 17 for association with meat quality or growth traits. The testing may involve genotyping DNA from animals, and possibly be used as a genetic marker for the same in a given group, population or species, with respect to the polymorphic marker and analyzing the genotyping data for association with meat quality or growth traits using methods described herein and/or known to those of skill in the art.

Candidate Gene Approach

The candidate gene approach typically takes into account knowledge of biological processes of a disease as a basis for selecting genes that encode proteins that could be envisioned to be involved in the biological processes. For example, reasonable candidate genes for blood pressure disorders could be proteins and enzymes involved in the renin-angiotensin system. Candidate genes can be evaluated genetically as possible disease genes by linkage and/or association studies of markers in the candidate gene region.

Methods of Identifying a Candidate Meat Ouality, Fatness and/or Growth Gene

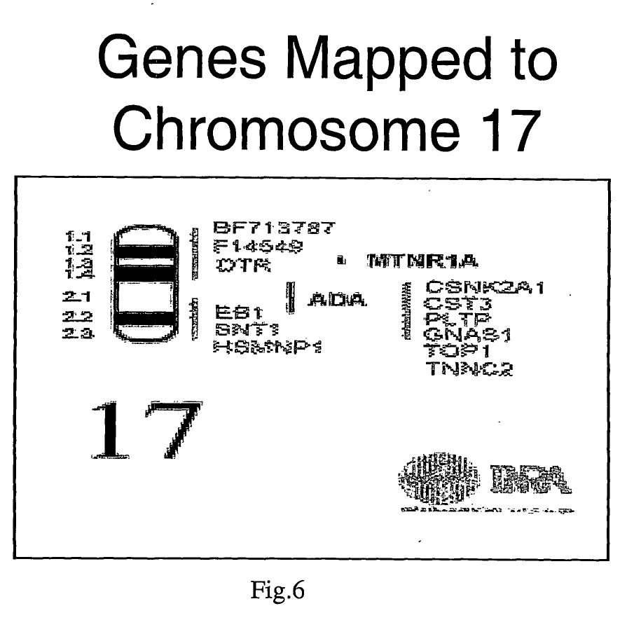

The methods of identifying a candidate meat quality, fatness and/or growth gene include a step of selecting a gene on chromosome 17 that is or encodes a product that has one or more properties relating to one or more phenomena in meat quality, fatness or growth. FIG. 6 provides a list of many of the genes that are located on chromosome 17. Additional genes that have been mapped to chromosome 17 are also known. Thus, genes on chromosome 17 may be evaluated as possible candidate genes on the basis of, for example, knowledge of the functions of the genes or products thereof and/or their occurrence or alteration in meat quality and growth.

Properties Relating to Phenomena in Meat Quality, Fatness and Growth

In the methods of identifying a candidate meat quality and growth gene provided herein, a gene on chromosome 17, and, in particular embodiments, on particular regions of chromosome 17 as described herein, are selected that is or encodes a product that has properties relating to one or more phenomena in meat quality and growth. The properties may be any aspect or feature of the gene or gene product, including but not limited to its physical composition (e.g., nucleic acids, amino acids, peptides and proteins), functional attributes (e.g., enzymatic capabilities, such as an enzyme catalyst, inhibitory functions, such as enzyme inhibition, antigenic properties, and binding capabilities, such as a receptor or ligand), cellular location(s), expression pattern (e.g., expression in the cells and tissues associated therewith) and/or interactions with other compositions.

The properties of the gene or gene product that are selected for in the methods of identifying a candidate meat quality, fatness and growth gene are those that relate to one or more phenomena in meat quality and growth. Such phenomena, which have been widely described and are known to those of skill in the art, are numerous and include morphological, structural, biological and biochemical occurrences. As described herein, the effect on meat quality may be demonstrated through the use of a particular identifier, such as pH or drip loss.

Candidate Genes of the Present Invention

Generally, in a candidate gene approach to the identification of a trait gene using association analysis of polymorphic markers, one or a few markers around or within candidate trait genes, particularly those with hypothesized functional importance, are genotyped in a few hundred case and control animals.

The specific characteristics of the associated allele with respect to a candidate gene function usually gives further insight into the relationship between the associated allele and the trait (causal or in linkage disequilibrium). If the evidence indicates that the associated allele within the candidate gene is most probably not the trait-causing allele but is in linkage disequilibrium with the real trait-causing allele, then the trait-causing allele can be found by sequencing the vicinity of the associated marker, and performing further association studies with the polymorphisms that are revealed in an iterative manner.

The Inventors of this invention have applied in part the candidate gene approach to meat quality, fatness and growth traits of the pig. The number of genes that are known to date that control meat quality and growth rates in pigs are small but their individual effects are, in most cases large. Often, this is due to the observation of the large effects that a polymorphism or mutation has on an animal's function. From such genes and others which seemed to be good candidates, the Inventors selected their candidate genes as disclosed herein. The candidate gene analysis clearly provides a short-cut approach to the identification of genes and gene polymorphisms related to a particular phenotypic trait when the candidate gene plays a plausible role in a biological or physiological pathway of the candidate gene. The basis of mutational effects on a trait in humans or mouse, suggests a role for the same gene in corresponding traits in livestock.

According to the invention, PKIG, MMP9, PTPN1, ATP9A, CYP24A1, DOK5, MC3R, AURKA, SPO11, RAE1, PCK1, RAB22A, GNAS, CTSZ, and PPP1R3D genes have all been identified as major effect genes and variability in these genes have been shown associated with the phenotypic traits of meat production or growth traits in animals, particularly pigs. Thus, screening methods may be developed for variation within or linked to these genes that are predictive of phenotypic variation.

Oligonucleotides were used in the PCR amplification of genomic DNA for sequences prior to design of specific oligonucleotides for single-nucleotide polymorphism (SNP) detection and genotyping. PCR conditions are exemplified in the Examples section.

The detection of the polymorphism(s) was carried out by restriction fragment length polymorphism detection. Genotyping for PKIG, MMP9, PTPN1, ATP9A, CYP24A1, DOK5, MC3R, AURKA, SPO11, RAE1, PCK1, RAB22A, GNAS, CTSZ, and PPP1R3D were based on the presence or absence of a restriction site at the polymorphic sites in PCR-amplified DNA fragments (PCR-RFLP). The genotypes were identified according to the resolved products on an electrophoretic gel.

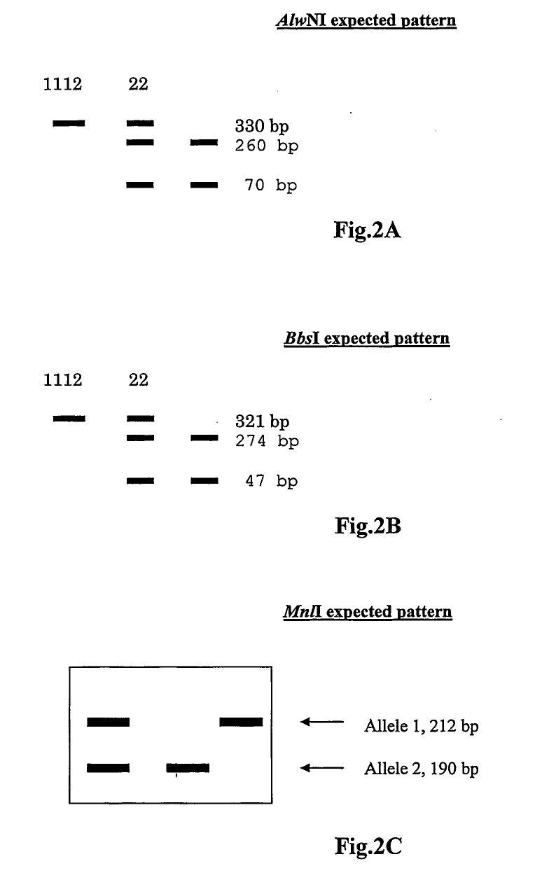

A mutation was detected on exon 2 of the porcine CTSZ gene depicted in SEQ ID NO:______. The RFLP detects an A/G substitution that causes an amino acid change (a lysine to an arginine). Digestion of an amplified CTSZ fragment with Alw NI resulted in an RFLP depicted in FIG. 2A. Homozygous allele 1 genotype generated a 330 base pair (bp) restriction fragment, while homozygous allele 2 genotype generated a 260 and 206 bp restriction fragment. Heterozygous 12 genotype showed all three fragments, 330, 260, and 70 bp.

A T/C substitution was detected on intron 7 of GNAS depicted in SEQ ID NO:______. The RFLP detects a T/C substitution in the coding region of exon 1, but does not cause an amino acid change. Digestion with Bbs I resulted in an RFLP depicted in FIG. 2B. Homozygous allele 1 genotype generated a 321 base pair (bp) restriction fragment, while homozygous allele 2 genotype generated a 274 and 47 bp restriction fragment. Heterozygous 12 genotype showed all three fragments, 321, 274, and 47 bp.

A T/C substitution was detected in the coding region of exon 1 in MC3R, but this mutation did not cause an amino acid change. Table 18 shows the various other polymorphisms identified.

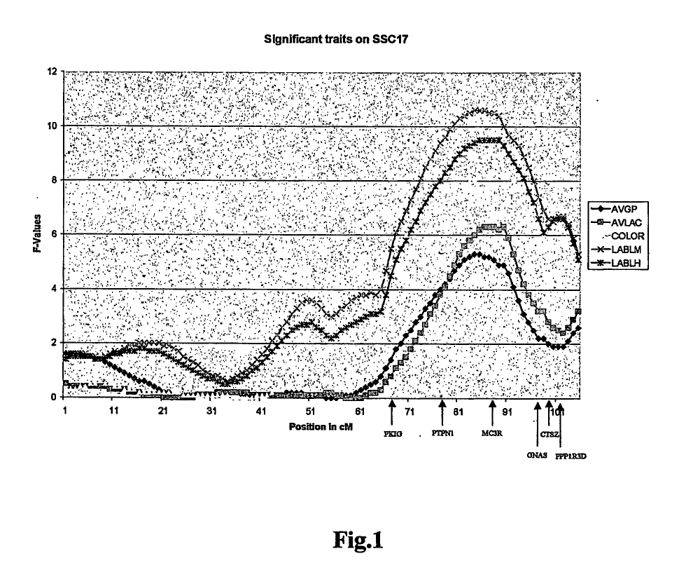

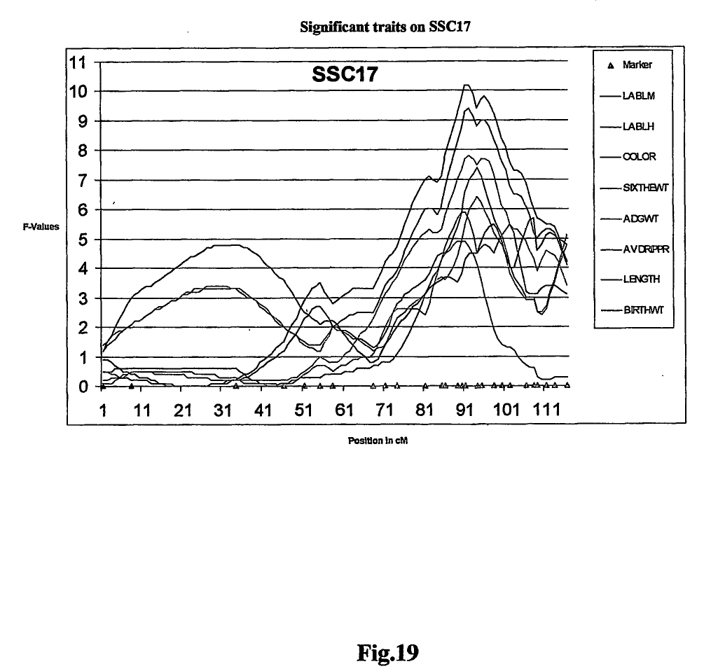

PKIG, MMP9, PTPN1, ATP9A, CYP24A1, DOK5, MC3R, AURKA, SPO11, RAE1, PCK1, RAB22A, GNAS, CTSZ, and PPP1R3D were mapped underneath the SSC17 QTL peaks for the above-mentioned traits. These QTL peaks include the regions on SSC17 that go approximately from 80 to 100 cM. The position of the genes on the original map is as follows: PKIG maps to about 66.8 cM, PTPN1 to about 77.4 cM, MC3R to about 88.5 cM, GNAS to about 96.2 cM, CTSZ to about 97.2 cM, and PPP1R3D to about 101.3 cM (FIG. 1). This map has been more specifically detailed according to the invention, see FIG. 19.

Any method of identifying the presence or absence of these polymorphisms may be used, including for example single-strand conformation polymorphism (SSCP) analysis, base excision sequence scanning (BESS), RFLP analysis, heteroduplex analysis, denaturing gradient gel electrophoresis, and temperature gradient electrophoresis, allelic PCR, ligase chain reaction direct sequencing, mini sequencing, nucleic acid hybridization, micro-array-type detection of a major effect gene or allele, or other linked sequences of the same. Also within the scope of the invention includes assaying for protein conformational or sequences changes, which occur in the presence of this polymorphism. The polymorphism may or may not be the causative mutation but will be indicative of the presence of this change and one may assay for the genetic or protein bases for the phenotypic difference. Based upon detection of there markers allele frequencies may be calculated for a given population to , determine differences in allele frequencies between groups of animals, i.e. the use of quantitative genotyping. This will provide for the ability to select specific populations for associated traits.

In general, the polymorphisms used as genetic markers of the present invention find use in any method known in the art to demonstrate a statistically significant correlation-between a genotype and a phenotype.

The invention therefore, comprises in one embodiment, a method of identifying an allele that is associated with meat quality traits. The invention also comprises methods of determining a genetic region or marker which may be used to identify and select animals based upon their meat quality, fatness or growth traits. Yet another embodiment provides a method of identifying an animal for its propensity for growth, fatness or meat quality traits.

Also provided herein are method of detecting an association between a genotype and a phenotype, which may comprising the steps of a) genotyping at least one candidate gene-related marker in a trait positive population according to a genotyping method of the invention; b) genotyping the candidate gene-related marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination. Preferably, the candidate gene-related marker is present in one or more of SEQ ID NOs:______ to ______ and more preferably is selected from the group consisting of Alw NI, Bbs I, Dde I, Msp I, Nae I, Afl III, Alw NI, Bse RI, Taa I, Mse I, Bst UI, Bcc I, Taq I, Nae I, and MnlI. Each of said genotyping of steps a) and b) is performed separately on biological samples derived from each pig in said population or a subsample thereof. Preferably, the phenotype is a trait involving the growth, fatness and meat quality characteristics of an animal.

The invention described herein contemplates alternative approaches that can be employed to perform association studies: genome-wide association studies, candidate region association studies and candidate gene association studies. In a preferred embodiment, the markers of the present invention are used to perform candidate gene association studies. Further, the markers of the present invention may be incorporated in any map of genetic markers of the pig genome in order to perform genome-wide association studies. Methods to generate a high-density map of markers well known to those of skill in the art. The markers of the present invention may further be incorporated in any map of a specific candidate region of the genome (a specific chromosome or a specific chromosomal segment for example).

Association studies are extremely valuable as they permit the analysis of sporadic or multifactor traits. Moreover, association studies represent a powerful method for fine-scale mapping enabling much finer mapping of trait causing alleles than linkage studies. Once a chromosome segment of interest has been identified, the presence of a candidate gene such as a candidate gene of the present invention, in the region of interest can provide a shortcut to the identification of the trait causing allele. Polymorphisms used as genetic markers of the present invention can be used to demonstrate that a candidate gene is associated with a trait. Such uses are specifically contemplated in the present invention and claims.

Association Analysis

The general strategy to perform association studies using markers derived from a region carrying a candidate gene is to scan two groups of animals (case-control populations) in order to measure and statistically compare the allele frequencies of the markers of the present invention in both groups.

If a statistically significant association with a trait is identified for at least one or more of the analyzed markers, one can assume that: either the associated allele is directly responsible for causing the trait (the associated allele is the trait causing allele), or more likely the associated allele is in linkage disequilibrium with the trait causing allele. The specific characteristics of the associated allele with respect to the candidate gene function usually gives further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker.

Association studies are usually run in two successive steps. In a first phase, the frequencies of a reduced number of markers from the candidate gene are determined in the trait positive and trait negative populations. In a second phase of the analysis, the position of the genetic loci responsible for the given trait is further refined using a higher density of markers from the relevant region. However, if the candidate gene under study is relatively small in length, a single phase may be sufficient to establish significant associations.

Testing for Association

Methods for determining the statistical significance of a correlation between a phenotype and a genotype, in this case an allele at a marker or a haplotype made up of such alleles, may be determined by any statistical test known in the art and is with any accepted threshold of statistical significance being required. The application of particular methods and thresholds of significance are well with in the skill of the ordinary practitioner of the art.

Testing for association is performed in one way by determining the frequency of a marker allele in case and control populations and comparing these frequencies with a statistical test to determine if there is a statistically significant difference in frequency which would indicate a correlation between the trait and the marker allele under study. Similarly, a haplotype analysis is performed by estimating the frequencies of all possible haplotypes for a given set of markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used and many exist. Preferably the statistical test employed is a chi-square test with one degree of freedom. A P-value is calculated (the P-value is the probability that a statistic as large or larger than the observed one would occur by chance). Other methods involve linear models and analysis of variance techniques.

The following is a general overview of techniques which can be used to assay for the polymorphisms of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the major effect gene of the invention as described in Sambrook et al., supra. RNA can be total cellular RNA, mRNA, poly A+RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl ) in a buffer of 50 mM Tris-HCl (pH 8.3), 50 mM KCl 1.5 mM MgCl_{2,} 0.5% Tween 20, 0.5% NP40 supplemented with 100 μg/ml of proteinase K. After incubating at 56° C. for 2 hr. the cells are heated to 95° C for 10 min to inactivate the proteinase K and immediately moved to wet ice (snap-cool). If gross aggregates are present, another cycle of digestion in the same buffer should be undertaken. Ten pll of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl_{2,} 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 pi of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6 M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N. H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by-a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase MT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Salki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wild type allele.

Ligase Mediated Allele Detection Method

Target regions of a test subject's DNA can be compared with target. regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (TM). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at an particular locus can be differentiated using single-strand conformation polymorphism analysis, which identifies base differences by alteration in electrophoretic migration of single stranded PCR products, as described in Orita et al., Proc. Nat. Acad. Sci. 85:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

Non-Gel Systems

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e. there is a mismatch of some form; the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay which includes isothermic amplification that relies on a catalytic release of fluorescence. See Third Wave Technology at www.twt.com.

Non-PCR Based DNA Diagnostics

The identification of a DNA sequence linked to an allele sequence can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with 32P or 35S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a porcine chromosome where one of the major effect genes .resides, and thus defminig a genetic marker linked to one of the major effect genes, including a restriction fragment length polyinorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Indeed in some situations it may be preferable to use combinations of markers giving specific haplotypes. Additional enzymes, constructed probes and primers can be determined through routine experimentation, combined with the teachings provided and incorporated herein.

According to one embodiment of the invention, polymorphisms in major effect genes have been identified which have an association with growth and meat quality. The presence or absence of the markers, in one embodiment may be assayed by PCR RFLP analysis using the restriction endonucleases and amplification primers may be designed using analogous human, pig or other of the sequences due to the high homology in the region surrounding the polymorphisms, or may be designed using known sequences (for example, human) as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, Fourth Edition, John Wiley and Sons 1999. The following is a brief description of primer design.

### Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above (see prediction of Nucleic Acid Structure). If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate animal DNA, genetically type individual animals, and detect genetic differences in animals. In particular, a sample of animal genomic DNA may be evaluated by reference to one or more controls to determine if a polymorphism in one of the sequences is present. Preferably, RFLP analysis is performed with respect to the animal's sequences, and the results are compared with a control. The control is the result of a RFLP analysis of one or both of the sequences of a different animal where the polymorphism of the animal gene is known. Similarly, the genotype of an animal may be determined by obtaining a sample of its genomic DNA, conducting RFLP analysis of the gene in the DNA, and comparing the results with a control. Again, the control is the result of RFLP analysis of one of the sequences of a different animal. The results genetically type the animal by specifying the polymorphism(s) in its gene. Finally, genetic differences among animals can be detected by obtaining samples of the genomic DNA from at least two animals, identifying the presence or absence of a polymorphism in one of the nucleotide sequences, and comparing the results.

These assays are useful for identifiing the genetic markers relating to growth and meat quality, as discussed above, for identifying other polymorphisms in the same genes or alleles that may be correlated with other characteristics, and for the general scientific analysis of animal genotypes and phenotypes.

One of skill in the art, once a polymorphism has been identified and a correlation to a particular trait established will understand that there are many ways to genotype animals for this polymorphism. The design of such alternative tests merely represents optimization of parameters known to those of skill in the art and is intended to be within the scope of this invention as fully described herein.

In accordance with the present invention there may be employed conventional molecular biology, microbiology, and recombinant DNA techniques within the skill of the art. Such techniques are explained fully in the literature. See, e.g., Maniatis, Fritsch & Sambrook, Molecular Cloning: A Laboratory Manual (1982); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription and Translation (B. D. Hames & S. J. Higgins eds. (1984)); Animal Cell Culture (R. I. Freshney, ed. (1986)); Immobilized Cells And Enzymes (IRL Press, (1986)); B. Perbal, A Practical Guide To Molecular Cloning, (1984).

The following examples serves to better illustrate the invention described herein and are not intended to limit the invention in any way. Those skilled in the art will recognize that there are several different parameters which may be altered using routine experimentation and which are intended to be within the scope of this invention.

### EXAMPLE 1

### Cathepsin Z (CTSZ) PCR-RFLP Test AlwNI Polymorphism

Primers

(SEQ ID NO: )

CT04F: 5′ GGC ATT TGG GGC ATC TGG G 3′

(SEQ ID NO: )

CT04R: 5′ ACT GGG GGA TGT GCT GGT T 3′

PCR Conditions:

Mix 1:

| | |
|---|---|
| 10X Promega Buffer | 1.0 μL |
| 25 mM MgCl₂ | 0.4 μL |
| dNTPs mix (2 mM) | 0.5 μL |
| 25 pmol/μL CT04F | 0.1 μL |
| 25 pmol/μL CT04R | 0.1 μL |
| dd sterile H₂0 | 6.83 μL |
| Taq Polymerase (5 U/μL) | 0.07 μL |
| genomic DNA (12.5 ng/μL) | 1.0 μL |

Combined 10 μL of Mix 1 and DNA in a reaction tube and overlaid with mineral oil. The following PCR program was ran: 94° C. for 3 min; 35 cycles of 94° C. for 30 sec, 62° C. 30 sec, and 72° C. 30 sec; followed by a final extension at 72° C. for 5 min. Checked 4 μL of the PCR reaction on a standard 1% agarose gel to confirm amplification success and clean negative control. The product size was approximately 330 base pairs. Digestion was performed using the following procedure:

| AlwNI Digestion Reaction | 10 μL reaction |
|---|---|
| PCR product | 5.0 μL |
| 10X NEB Buffer 4 | 1.0 μL |
| AlwNI enzyme (10 U/μL) | 0.5 μL |
| dd sterile H₂0 | 3.5 μL |

Made a cocktail with the buffer, enzyme and water. Added 5 μL to each reaction tube containing the DNA. Incubated at 37° C. at least 4 hours, although the digestion overnight was preferred. Mixed 4 μL of loading dye with 6 μL of the digested PCR product and loaded the total volume on a 3% agarose gel. The AlwNI pattern expected is shown in FIG. 2A.

### EXAMPLE 2

### GNAS PCR-RFLP Test BbsI Polymorphism

Primers

(SEQ ID NO: )

GN03F: 5′ AAG CAG GCT GAC TAC GTG 3′

(SEQ ID NO: )

GN03R: 5′ TCA CCA CAA GGG CTA CCA 3′

PCR Conditions:

Mix 1:

| | |
|---|---|
| 10X Promega Buffer | 1.0 μL |
| 25 mM MgCl₂ | 0.8 μL |
| dNTPs mix (2 mM) | 0.5 μL |
| 25 pmol/μL GN03F | 0.1 μL |
| 25 pmol/μL GN03R | 0.1 μL |
| dd sterile H₂0 | 6.43 μL |
| Taq Polymerase (5 U/μL) | 0.07 μL |
| genomic DNA (12.5 ng/μL) | 1.0 μL |

Combined 10 μL of Mix 1 and DNA in a reaction tube and overlaid with mineral oil. The following PCR program was ran: 94° C. for 3 min; 35 cycles of 94° C. for 30 sec, 60° C. 30 sec, and 72° C. 30 sec; followed by a final extension at 72° C. for 5 min. Checked 4 μL of the PCR reaction on a standard 1% agarose gel to confirm amplification success and clean negative control. Product size was approximately 321 base pairs. Digestion was performed using the following procedure:

| BbsI Digestion Reaction | 10 μL reaction |
|---|---|
| PCR product | 4.0 μL |
| 10X NEB Buffer 2 | 1.0 μL |
| BbsI enzyme (5 U/μL) | 0.5 μL |
| dd sterile H₂0 | 4.5 μL |

Made a cocktail with the buffer, enzyme and water. Added 6 μL to each reaction tube containing the DNA. Incubated at 37° C. at least 4 hours, although digestion overnight was preferred. Mixed 4 μL of loading dye with 6 μL of the digested PCR product and loaded the total volume on a 3% agarose gel. The BbsI expected pattern is shown in FIG. 2B.

### EXAMPLE 3

### MC3R PCR-RFLP Test MnlI Polymorphism

Primers:

(SEQ ID NO: )

Forward: 5′ GCC TCC ATC TGC AAC CTC T 3′

(SEQ ID NO: )

Reverse: 5′ AGC ATG GCG AAG AAG ATG AC 3′

PCR Conditions:

Mix 1

| | |
|---|---|
| 10 X PCR Buffer | 1.0 μl |
| MgCl₂ (25 mM) | 0.6 μl |
| dNTPs (2.5 mM) | 0.5 μl |
| Forward (25 pmol/μl) | 0.1 μl |
| Reverse (25 pmol/μl) | 0.1 μl |
| Taq Polymerase (5 U/μl) | 0.07 μl |
| ddH₂O | 7.63 μl |
| genomic DNA | 1.0 μl |

Combined the Mix 1 and DNA in a PCR reaction tube and overlaid the mix with mineral oil. The following PCR program was ran: 94° C. for 3 min; 36 cycles of 94° C. for 30 sec, 54° C. for 1 min, and 72° C. for lmin 30 sec; followed by a final extension at 72° C. for 10 min. Checked 2 μl of the PCR on a 1.6% agarose gel to confirm amplification success and the desirable clean result in the negative control.

Digestion can be Performed by the Following Procedures:

MnlI Digestion Reaction:

| | |
|---|---|
| PCR product | 4.0 μl |
| NE Buffer 2 | 1.0 μl |
| BSA (10 mg/ml) | 0.1 μl |
| MnlI (20 U/μl) | 0.2 μl |
| ddH₂O | 4.7 μl |

Made a cocktail of the PCR product, buffer, enzyme and water. Incubated for at least 4 hours, although overnight at 37° C. was preferred. Mixed the digest with loading dye (2:5) and ran on a 3 % NuSieve agarose gel. The MnlI expected pattern is shown in FIG. 2C.

### EXAMPLE 4

### Associations Between CTSZ, GNAS and MC3R Genotypes and Several Economic Traits Were Investigated in a Berkshire×Yorkshire Cross (Tables 3, 4 and 5, Respectively).

TABLE 3
Association of CTSZ genotypes with several meat quality traits in a pig
resource population.

| | CTSZ Genotype | | | |
|---|---|---|---|---|
| Trait | 11 | 12 | 22 | P-value |
| Color | 3.07 ± 0.07 e, i | 3.22 ± 0.04 f, c | 3.31 ± 0.03 j, d | 0.0049 |
| LabLH | 47.99 ± 0.50 ai | 47.25 ± 0.29 be | 46.52 ± 0.27 jf | 0.0055 |
| LabLM | 23.10 ± 0.47 ai | 22.37 ± 0.27 be | 21.62 ± 0.25 jf | 0.0030 |
| Av. Glyco. Pot. | 106.65 ± 2.44 a | 105.96 ± 1.33 a | 103.84 ± 1.21 b | 0.2987 |
| Av. Lactate | 88.36 ± 1.89 | 88.16 ± 1.02 a | 86.51 ± 0.93 b | 0.3314 |
| Lumbar Backfat | 3.71 ± 0.11 ae | 3.59 ± 0.07 bc | 3.49 ± 0.07 fd | 0.0750 |
| Av. Drip Loss | 6.34 ± 0.28 ce | 5.83 ± 0.16 da | 5.63 ± 0.15 fb | 0.0449 |
| Flavor score | 2.18 ± 0.23 i | 2.89 ± 0.12 j | 2.93 ± 0.11 j | 0.0072 |
| Juiciness score | 6.31 ± 0.20 e | 5.80 ± 0.10 fi | 6.20 ± 0.10 j | 0.0034 |
| Cooking Loss | 17.91 ± 0.58 e | 19.29 ± 0.30 f | 18.36 ± 0.28 e | 0.0197 |
| Tenderness score | 7.58 ± 0.18 e | 7.75 ± 0.10 c | 7.95 ± 0.10 fd | 0.0696 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

The results presented on Table 3 indicate that CTSZ genotypes are associated with the five meat quality QTL traits. The strongest associations were with color, LabLH and LabLM. In addition, associations with other meat quality traits, such as average drip loss and tenderness, were also detected, fact that strengthens the potential use of this marker in the selection of pigs for improved meat quality.

TABLE 4
Association of GNAS genotypes with several meat quality traits in a pig
resource population.

GNAS Genotype

| Trait | 11 | 12 | 22 | P-value |
|---|---|---|---|---|
| Color | 3.11 ± 0.05 ie | 3.29 ± 0.03 j | 3.26 ± 0.05 f | 0.0098 |
| LabLH | 47.81 ± 0.41 ei | 46.90 ± 0.27 fa | 46.50 ± 0.37 jb | 0.0237 |
| LabLM | 22.88 ± 0.38 ei | 22.00 ± 0.25 fa | 21.60 ± 0.35 jb | 0.0195 |
| Av. Glyco. Pot. | 107.44 ± 1.90 a | 104.63 ± 1.18 b | 104.02 ± 1.64 b | 0.2944 |
| Av. Lactate | 89.22 ± 1.48 a | 87.02 ± 0.92 b | 86.74 ± 1.28 b | 0.3163 |
| Av. Drip Loss | 6.35 ± 0.22 Ie | 5.67 ± 0.14 j | 5.66 ± 0.20 f | 0.0079 |
| Tenderness score | 7.56 ± 0.14 ec | 7.88 ± 0.09 f | 7.89 ± 0.13 d | 0.0803 |
| WHC | 0.22 ± 0.013 ae | 0.20 ± 0.008 ba | 0.18 ± 0.012 fb | 0.0753 |
| Chew Score | 2.69 ± 0.11 e | 2.41 ± 0.07 f | 2.36 ± 0.10 f | 0.0312 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

In accordance with the results determined for CTSZ, the GNAS genotypes were also found to be associated with the five QTL meat quality traits on SSC17. The results for this marker indicate that the strongest associations were detected with color, LabLH and LabLM, which is inline with the effect of the CTSZ genotypes. Other meat quality traits (average drip loss, tenderness score) were also affected by this marker, further indicating the usefulness of these genetic markers mapped on this specific region of SSC17 as tools to select pigs for higher meat quality.

TABLE 5
Association of MC3R genotypes with several growth, fatness and
meat quality traits in a pig resource population.

| | MC3R Genotype | | |
|---|---|---|---|
| Trait | 11 | 12 | P-value |
| Color | 3.26 ± 0.03 | 3.21 ± 0.06 | 0.4415 |
| LabLH | 47.00 ± 0.23 | 46.70 ± 0.45 | 0.4971 |
| LabLM | 22.10 ± 0.21 | 21.83 ± 0.42 | 0.5311 |
| Av. Glyco. Pot. | 105.71 ± 1.06 e | 101.23 ± 2.14 f | 0.0379 |
| Av. Lactate | 87.83 ± 0.81 c | 84.96 ± 1.66 d | 0.0887 |
| Birth Weight | 1.52 ± 0.04 i | 1.63 ± 0.05 j | 0.0077 |
| Carcass weight | 87.22 ± 0.16 c | 86.70 ± 0.31 d | 0.0893 |
| Av. Daily Gain on | 0.685 ± 0.006 e | 0.703 ± 0.009 f | 0.0152 |
| Test | | | |
| Av. Backfat | 3.25 ± 0.05 c | 3.39 ± 0.08 d | 0.0643 |
| Lumbar Backfat | 3.53 ± 0.06 c | 3.69 ± 0.10 d | 0.0618 |
| Tenthrib Backfat | 3.08 ± 0.06 c | 3.25 ± 0.10 d | 0.0511 |
| Loin Eye Area | 36.26 ± 0.55 m | 33.94 ± 0.76 n | 0.0002 |
| Fiber Type II Ratio | 0.99 ± 0.04 i | 1.32 ± 0.10 j | 0.0016 |
| Av. Glycogen Content | 8.92 ± 0.17 c | 8.17 ± 0.40 d | 0.0688 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

MC3R genotypes did not present any large effects on three of the SSC17 QTL traits for meat quality (color, LabLH and LabLM). However, a more significant effect of this marker on average glycolytic potential and average lactate was detected, when compared with the influence of CTSZ and GNAS genotypes on these two traits. Furthermore, MC3R variants were strongly associated with several growth, fatness and carcass composition traits, which indicates that this marker can be used in the selection of pigs with improved meat quality and growth traits.

In addition to the studies conducted in the pig resource population, the effect of these three genes was also analyzed in several commercial pure and synthetic lines (Landrace, Large White and Synthetic). The results are indicated on tables 6 to 15.

TABLE 6
Analysis of CTSZ effect on meat quality and production traits in a commercial
Landrace population.

| | | LSmeans (s.e.) | | | |
|---|---|---|---|---|---|
| Trait | Mean (s.e.) | 11 | 12 | 22 | P-value |
| dirtywt | 245.1 (0.84) | 244.5 (1.79) | 245.9 (1.31) | 245.6 (1.97) | 0.76 |
| hcw | 195.1 (0.67) | 195.3 (1.49) | 195.3 (1.10) | 196.5 (1.66) | 0.78 |
| ccw | 192.7 (0.69) | 192.4 (1.52) | 192.9 (1.10) | 193.9 (1.69) | 0.79 |
| l_binwt | 20.97 (0.10) | 21.10 (0.19)a | 20.85 (0.14)b | 20.96 (0.20) | 0.42 |
| l_blswt | 7.20 (0.05) | 7.18 (0.09) | 7.15 (0.07) | 7.15 (0.10) | 0.94 |
| loinminl | 44.42 (0.14) | 44.75 (0.32)e | 44.10 (0.24)fa | 43.67 (0.36)fb | 0.04 |
| loinmina | 6.70 (0.06) | 6.76 (0.13) | 6.73 (0.10) | 6.84 (0.15) | 0.80 |
| loinminb | 2.92 (0.06) | 3.09 (0.09) | 2.99 (0.07) | 3.01 (0.10) | 0.60 |
| japcs | 3.31 (0.03) | 3.31 (0.08)a | 3.39 (0.06) | 3.43 (0.08)b | 0.50 |
| marbling | 1.72 (0.03) | 1.68 (0.06) | 1.71 (0.04) | 1.76 (0.06) | 0.62 |
| firmness | 2.78 (0.07) | 2.92 (0.10) | 2.90 (0.07) | 2.88 (0.10) | 0.96 |
| loinpH | 5.69 (0.01) | 5.69 (0.01) | 5.70 (0.01) | 5.69 (0.01) | 0.81 |
| h_binwt | 22.93 (0.15) | 22.68 (0.27) | 22.93 (0.19) | 22.75 (0.28) | 0.65 |
| h_blswt | 4.37 (0.03) | 4.41 (0.06) | 4.36 (0.04) | 4.38 (0.06) | 0.72 |
| hamminl | 47.27 (0.22) | 47.16 (0.51)c | 48.12 (0.38)de | 46.68 (0.53)f | 0.02 |
| hammina | 8.61 (0.10) | 8.73 (0.21) | 8.74 (0.15) | 8.80 (0.22) | 0.97 |
| hamminb | 4.29 (0.10) | 4.25 (0.19)c | 4.65 (0.14)de | 4.19 (0.20)f | 0.03 |
| hampH | 5.67 (0.01) | 5.70 (0.02)a | 5.69 (0.01)a | 5.68 (0.02)b | 0.39 |
| dripprct | 2.76 (0.09) | 2.62 (0.20) | 2.56 (0.14) | 2.48 (0.21) | 0.88 |
| hprofat | 12.95 (0.12) | 12.92 (0.27) | 13.08 (0.20) | 13.04 (0.29) | 0.86 |
| hpromeat | 53.20 (0.58) | 53.89 (0.80) | 54.08 (0.62) | 53.77 (0.87) | 0.93 |
| hprorib | 13.05 (0.25) | 12.23 (0.58) | 12.79 (0.40)a | 11.93 (0.57)b | 0.30 |
| LMprct | 46.79 (0.08) | 47.07 (0.21) | 46.91 (0.15) | 46.94 (0.21) | 0.74 |
| gcaloc_f | 12.99 (0.15) | 13.32 (0.35)ac | 12.76 (0.26)b | 12.46 (0.39)d | 0.17 |
| gcendwt | 112.7 (0.33) | 113.3 (0.71)a | 112.4 (0.53)b | 111.7 (0.80)b | 0.25 |
| gcdays | 158.9 (0.71) | 155.8 (0.99)ae | 157.2 (0.78)b | 159.3 (1.19)af | 0.07 |
| gcldg | 674.5 (2.10) | 668.3 (4.39)a | 664.6 (3.35) | 660.8 (5.17)b | 0.49 |
| gctdg | 898.7 (3.77) | 899.5 (7.75)a | 891.8 (5.86) | 887.1 (9.12)b | 0.50 |
| gcus_md | 60.72 (0.38) | 59.61 (0.77)e | 61.38 (0.56)fa | 60.18 (0.91)b | 0.07 |
| dirtywt | 245.1 (0.84) | 244.5 (1.79) | 245.9 (1.31) | 245.6 (1.97) | 0.76 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 7
Analysis of CTSZ effect on meat quality and production traits in a commercial
Large White population.

| | | LSmeans (s.e.) | | | |
|---|---|---|---|---|---|
| Trait | Mean (s.e.) | 11 | 12 | 22 | P-value |
| dirtywt | 236.7 (1.01) | 238.1 (2.77)a | 236.6 (1.98)a | 234.0 (2.21)b | 0.32 |
| hcw | 188.2 (0.77) | 189.8 (2.10)a | 188.8 (1.50)a | 186.8 (1.68)b | 0.33 |
| ccw | 186.0 (0.81) | 189.0 (2.22)ac | 186.1 (1.51)b | 184.6 (1.73)d | 0.20 |
| l_binwt | 19.82 (0.13) | 19.34 (0.27)a | 19.76 (0.21)b | 19.73 (0.22)b | 0.37 |
| l_blswt | 6.70 (0.05) | 6.59 (0.10) | 6.66 (0.08) | 6.64 (0.09) | 0.79 |
| loinminl | 44.79 (0.17) | 44.61 (0.46) | 44.95 (0.33) | 44.73 (0.37) | 0.72 |
| loinmina | 7.35 (0.09) | 7.18 (0.24)a | 6.91 (0.17)b | 7.00 (0.19) | 0.56 |
| loinminb | 3.08 (0.07) | 3.18 (0.16) | 3.11 (0.11)a | 3.26 (0.13)b | 0.45 |
| japcs | 3.35 (0.05) | 3.35 (0.12) | 3.47 (0.09)a | 3.35 (0.10)b | 0.39 |
| marbling | 1.89 (0.05) | 2.06 (0.10)c | 1.86 (0.08)da | 1.99 (0.08)b | 0.11 |
| firmness | 2.41 (0.07) | 2.73 (0.15) | 2.85 (0.12)a | 2.64 (0.13)b | 0.27 |
| loinpH | 5.69 (0.01) | 5.67 (0.02) | 5.68 (0.01) | 5.68 (0.02) | 0.95 |
| h_binwt | 23.12 (1.24) | 21.70 (0.25)a | 21.97 (0.20)b | 21.69 (0.21)a | 0.28 |
| h_blswt | 3.92 (0.04) | 3.81 (0.10)a | 3.99 (0.08)b | 3.86 (0.08)a | 0.17 |
| hamminl | 45.27 (0.31) | 46.74 (0.79)a | 46.04 (0.60) | 45.53 (0.65)b | 0.41 |
| hammina | 9.49 (0.13) | 9.82 (0.35)ce | 9.16 (0.26)d | 8.99 (0.28)f | 0.12 |
| hamminb | 4.34 (0.15) | 5.20 (0.32)e | 4.47 (0.24)f | 4.38 (0.27)f | 0.08 |
| hampH | 5.74 (0.02) | 5.64 (0.03)ac | 5.69 (0.02)b | 5.70 (0.02)d | 0.16 |
| dripprct | 2.35 (0.11) | 2.40 (0.33) | 2.53 (0.24) | 2.29 (0.25) | 0.63 |
| hprofat | 14.07 (0.15) | 13.80 (0.45)a | 14.30 (0.32)b | 13.99 (0.35) | 0.42 |
| hpromeat | 50.39 (0.71) | 50.53 (1.09)a | 51.82 (0.77)b | 50.61 (0.85)a | 0.25 |
| hprorib | 13.36 (0.36) | 14.61 (0.95)a | 13.07 (0.74)b | 13.29 (0.79)b | 0.35 |
| LMprct | 46.14 (0.10) | 45.92 (0.29)a | 46.35 (0.21)b | 46.32 (0.23)b | 0.40 |
| gcaloc_f | 13.45 (0.18) | 13.64 (0.51) | 13.48 (0.37) | 13.82 (0.41) | 0.67 |
| gcendwt | 109.2 (0.36) | 110.0 (1.01)ae | 108.6 (0.72)bc | 107.3 (0.82)fd | 0.047 |
| gcdays | 170.7 (0.76) | 161.0 (1.81)c | 162.8 (1.36)a | 164.8 (1.52)db | 0.15 |
| gcldg | 644.9 (2.34) | 650.6 (5.50)a | 646.4 (3.76)a | 640.7 (4.37)b | 0.23 |
| gctdg | 841.4 (3.85) | 853.5 (9.49)ae | 840.9 (6.42)b | 829.5 (7.51)af | 0.08 |
| gcus_md | 59.07 (0.39) | 58.73 (1.03) | 59.02 (0.62) | 59.47 (0.74) | 0.78 |
| dirtywt | 236.7 (1.01) | 238.1 (2.77)a | 236.6 (1.98)a | 234.0 (2.21)b | 0.32 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 8
Analysis of CTSZ effect on meat quality and production traits in a Synthetic
commercial population.

| | | LSmeans (s.e.) | | | |
|---|---|---|---|---|---|
| Trait | Mean (s.e.) | 11 | 12 | 22 | P-value |
| Dirtywt | 248.6 (1.54) | 248.5 (5.40)a | 244.0 (2.59) | 241.2 (3.01)b | 0.41 |
| Hcw | 204.3 (1.16) | 204.5 (3.90)a | 200.3 (1.85) | 199.7 (2.18)b | 0.53 |
| Ccw | 202.4 (1.19) | 202.6 (3.94)a | 198.2 (1.91)b | 198.7 (2.28) | 0.56 |
| l_binwt | 22.55 (0.22) | 22.85 (0.53)a | 22.28 (0.23)b | 22.57 (0.34) | 0.51 |
| l_blswt | 8.17 (0.09) | 8.35 (0.25) | 8.10 (0.12) | 8.26 (0.17) | 0.52 |
| Loinminl | 45.06 (0.20) | 43.72 (0.69)a | 44.81 (0.34)b | 44.66 (0.40)b | 0.32 |
| Loinmina | 6.78 (0.10) | 7.20 (0.34) | 6.92 (0.18) | 7.07 (0.21) | 0.62 |
| Loinminb | 2.90 (0.08) | 2.86 (0.19)a | 3.05 (0.09) | 3.14 (0.11)b | 0.39 |
| Japcs | 3.31 (0.07) | 3.12 (0.22)a | 3.45 (0.09)b | 3.32 (0.12) | 0.29 |
| Marbling | 2.16 (0.09) | 2.30 (0.23) | 2.23 (0.10) | 2.26 (0.12) | 0.96 |
| Firmness | 2.80 (0.15) | 3.62 (0.31)ea | 2.88 (0.14)fa | 3.23 (0.22)b | 0.048 |
| LoinpH | 5.74 (0.01) | 5.74 (0.04) | 5.74 (0.02) | 5.74 (0.02) | 0.98 |
| h_binwt | 25.57 (0.23) | 26.53 (0.56)ac | 25.60 (0.25)b | 25.38 (0.35)d | 0.18 |
| h_blswt | 5.18 (0.05) | 5.33 (0.18)a | 5.15 (0.08) | 5.04 (0.11)b | 0.34 |
| Hamminl | 47.65 (0.53) | 49.77 (1.63)c | 48.32 (0.75)c | 46.28 (0.97)d | 0.09 |
| Hammina | 8.53 (0.16) | 8.82 (0.57) | 8.50 (0.26) | 8.81 (0.33) | 0.67 |
| Hamminb | 4.09 (0.17) | 5.20 (0.44)ae | 4.52 (0.20)b | 4.22 (0.26)f | 0.14 |
| HampH | 5.69 (0.02) | 5.60 (0.06)a | 5.68 (0.03)b | 5.69 (0.03)b | 0.31 |
| Dripprct | 2.05 (0.14) | 2.44 (0.50) | 2.10 (0.23) | 2.35 (0.30) | 0.63 |
| Hprofat | 13.52 (0.20) | 13.64 (0.69) | 13.73 (0.33) | 13.33 (0.39) | 0.66 |
| Hpromeat | 62.84 (0.88) | 62.36 (2.05)a | 60.09 (0.96)b | 60.47 (1.12) | 0.57 |
| Hprorib | 15.15 (0.62) | 9.48 (2.54)e | 15.31 (0.92)fa | 17.33 (1.50)fb | 0.051 |
| LMprct | 47.54 (0.18) | 47.91 (0.67) | 47.30 (0.27) | 47.56 (0.46) | 0.62 |
| Gcaloc_f | 12.96 (0.25) | 13.85 (0.82)a | 13.68 (0.42)c | 12.72 (0.49)bd | 0.15 |
| Gcendwt | 113.7 (0.53) | 113.2 (1.69) | 112.5 (0.85) | 111.6 (0.99) | 0.61 |
| Gcdays | 163.6 (0.81) | 157.1 (2.71)a | 160.3 (1.44)b | 160.2 (1.65)b | 0.51 |
| Gcldg | 670.3 (3.06) | 677.5 (9.38) | 670.3 (4.92) | 667.0 (5.44) | 0.59 |
| Gctdg | 880.4 (4.97) | 886.4 (16.5) | 875.1 (8.39) | 870.9 (9.28) | 0.70 |
| Gcus_md | 67.11 (0.51) | 67.92 (1.47)ea | 64.86 (0.76)fa | 65.91 (0.88)b | 0.097 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 9
Overall analysis of CTSZ effect on meat quality and production traits in
commercial Landrace, Large White and Synthetic populations.

| | | LSmeans (s.e.) | | | |
|---|---|---|---|---|---|
| Trait | Mean (s.e.) | 11 | 12 | 22 | P-value |
| dirtywt | 243.1 (0.62) | 241.6 (1.53) | 242.1 (1.04)a | 240.6 (1.25)b | 0.51 |
| hcw | 194.7 (0.50) | 195.3 (1.21) | 194.7 (0.82) | 194.5 (1.00) | 0.86 |
| ccw | 192.6 (0.52) | 193.0 (1.25) | 192.3 (0.84) | 192.4 (1.04) | 0.84 |
| l_binwt | 20.88 (0.08) | 20.94 (0.16) | 20.79 (0.11)a | 20.97 (0.13)b | 0.36 |
| l_blswt | 7.21 (0.04) | 7.28 (0.07) | 7.26 (0.05) | 7.30 (0.06) | 0.81 |
| loinminl | 44.66 (0.09) | 44.81 (0.25)a | 44.63 (0.17) | 44.40 (0.21)b | 0.34 |
| loinmina | 6.92 (0.05) | 6.99 (0.12) | 6.94 (0.09) | 7.03 (0.10) | 0.69 |
| loinminb | 2.97 (0.04) | 3.26 (0.08) | 3.22 (0.05)a | 3.31 (0.06)b | 0.40 |
| japcs | 3.32 (0.02) | 3.30 (0.07)ca | 3.41 (0.05)d | 3.38 (0.06)b | 0.22 |
| marbling | 1.84 (0.03) | 1.94 (0.05) | 1.91 (0.04)c | 1.99 (0.05)d | 0.26 |
| firmness | 2.68 (0.05) | 3.06 (0.09) | 3.06 (0.06) | 2.99 (0.07) | 0.61 |
| loinpH | 5.70 (0.00) | 5.70 (0.01) | 5.70 (0.01) | 5.70 (0.01) | 0.96 |
| h_binwt | 23.39 (0.38) | 23.26 (0.19) | 23.43 (0.13) | 23.25 (0.16) | 0.50 |
| h_blswt | 4.36 (0.03) | 4.48 (0.05) | 4.49 (0.04)a | 4.44 (0.04)b | 0.57 |
| hamminl | 46.73 (0.18) | 47.33 (0.44)a | 47.70 (0.31)g | 46.62 (0.37)bh | 0.02 |
| hammina | 8.86 (0.07) | 8.99 (0.18) | 8.90 (0.13) | 8.87 (0.15) | 0.81 |
| hamminb | 4.27 (0.07) | 4.75 (0.16)a | 4.80 (0.11)e | 4.51 (0.14)bf | 0.12 |
| hampH | 5.69 (0.01) | 5.68 (0.01) | 5.69 (0.01) | 5.69 (0.01) | 0.76 |
| dripprct | 2.51 (0.06) | 2.47 (0.17) | 2.43 (0.11) | 2.45 (0.13) | 0.98 |
| hprofat | 13.41 (0.09) | 13.40 (0.24) | 13.59 (0.16) | 13.40 (0.19) | 0.50 |
| hpromeat | 54.13 (0.42) | 55.57 (0.65) | 55.71 (0.45) | 55.17 (0.53) | 0.60 |
| hprorib | 13.40 (0.20) | 13.54 (0.53) | 13.93 (0.37) | 13.84 (0.45) | 0.76 |
| LMprct | 46.70 (0.06) | 46.89 (0.18) | 46.77 (0.12) | 46.80 (0.15) | 0.75 |
| gcaloc_f | 13.13 (0.11) | 13.46 (0.28)ce | 12.94 (0.20)d | 12.69 (0.24)f | 0.051 |
| gcendwt | 111.8 (0.23) | 112.4 (0.58)ci | 111.4 (0.40)de | 110.3 (0.49)jf | 0.005 |
| gcdays | 163.6 (0.48) | 155.5 (1.03)ag | 157.0 (0.77)bc | 158.6 (0.90)hd | 0.02 |
| gcldg | 664.3 (1.45) | 667.0 (3.21)ae | 662.2 (2.08)b | 657.8 (2.64)af | 0.06 |
| gctdg | 875.1 (2.51) | 887.4 (5.57)cg | 877.0 (3.54)da | 869.6 (4.52)hb | 0.04 |
| gcus_md | 61.63 (0.26) | 60.44 (0.61) | 61.05 (0.42) | 60.89 (0.52) | 0.60 |

TABLE 10
Analysis of GNAS effect on meat quality and production traits in
commercial Landrace population.

| | | LSmeans (s.e.) | | | |
|---|---|---|---|---|---|
| Trait | Mean (s.e.) | 11 | 12 | 22 | P-value |
| ccw | 192.7 (0.69) | 191.9 (1.39)e | 193.0 (1.15)c | 196.9 (2.06)fd | 0.08 |
| dirtywt | 244.7 (0.83) | 244.3 (1.66)a | 245.1 (1.38)a | 248.4 (2.44)b | 0.31 |
| dripprct | 2.87 (0.09) | 2.74 (0.17) | 2.72 (0.14) | 2.82 (0.24) | 0.92 |
| firmness | 2.78 (0.07) | 2.96 (0.08)a | 2.83 (0.07)be | 3.06 (0.11)f | 0.07 |
| gcaloc_f | 12.94 (0.15) | 12.92 (0.32)a | 12.83 (0.27)a | 12.07 (0.47)b | 0.24 |
| gcdays | 158.9 (0.70) | 156.0 (0.92)c | 157.9 (0.80)d | 158.9 (1.48)d | 0.10 |
| gcendwt | 112.8 (0.33) | 112.8 (0.66) | 112.5 (0.56) | 112.5 (1.00) | 0.90 |
| gcldg | 674.1 (2.06) | 666.8 (3.96) | 665.1 (3.52) | 668.8 (6.32) | 0.83 |
| gctdg | 898.5 (3.69) | 895.4 (7.04) | 892.9 (6.23) | 900.4 (11.3) | 0.81 |
| gcus_md | 60.76 (0.38) | 60.21 (0.70)a | 61.19 (0.59)b | 60.19 (1.11) | 0.39 |
| h_binwt | 22.92 (0.16) | 22.85 (0.23) | 22.94 (0.19) | 22.84 (0.34) | 0.94 |
| h_blswt | 4.36 (0.03) | 4.44 (0.05)a | 4.37 (0.04)b | 4.40 (0.07) | 0.39 |
| hammina | 8.61 (0.10) | 8.77 (0.20) | 8.93 (0.17)a | 8.45 (0.28)b | 0.25 |
| hamminb | 4.35 (0.10) | 4.41 (0.17)a | 4.66 (0.14)be | 4.11 (0.24)bf | 0.07 |
| hamminl | 47.31 (0.22) | 47.67 (0.44)a | 48.12 (0.36)e | 46.69 (0.61)bf | 0.08 |
| hampH | 5.66 (0.01) | 5.69 (0.01) | 5.69 (0.01) | 5.67 (0.02) | 0.60 |
| hcw | 195.0 (0.67) | 194.7 (1.36)c | 195.1 (1.15)c | 199.0 (2.03)d | 0.13 |
| hprofat | 12.91 (0.12) | 12.76 (0.25)a | 13.07 (0.22)b | 13.11 (0.36) | 0.45 |
| hpromeat | 52.95 (0.57) | 53.14 (0.74)a | 53.95 (0.64)b | 53.03 (1.03) | 0.43 |
| hprorib | 13.17 (0.25) | 12.00 (0.53)c | 13.06 (0.44)da | 11.93 (0.71)b | 0.09 |
| japcs | 3.30 (0.03) | 3.27 (0.07)ca | 3.40 (0.06)d | 3.41 (0.10)b | 0.17 |
| l_binwt | 20.90 (0.12) | 20.97 (0.21) | 20.81 (0.18) | 21.01 (0.31) | 0.72 |
| l_blswt | 7.20 (0.05) | 7.18 (0.08)a | 7.21 (0.07)a | 7.04 (0.11)b | 0.30 |
| LMprct | 46.79 (0.08) | 47.03 (0.18)a | 46.92 (0.15)a | 46.67 (0.23)b | 0.35 |
| loinmina | 6.69 (0.06) | 6.71 (0.12) | 6.72 (0.10) | 6.93 (0.19) | 0.56 |
| loinminb | 2.94 (0.06) | 3.04 (0.08) | 2.96 (0.07) | 3.05 (0.12) | 0.57 |
| loinminl | 44.32 (0.14) | 44.48 (0.29)ca | 43.96 (0.25)d | 43.97 (0.43)b | 0.21 |
| loinpH | 5.69 (0.01) | 5.69 (0.01) | 5.70 (0.01)a | 5.68 (0.02)b | 0.46 |
| marbling | 1.71 (0.03) | 1.66 (0.05)a | 1.73 (0.04)b | 1.66 (0.08) | 0.35 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 11
Analysis of GNAS effect on meat quality and production traits in
commercial Synthetic population.

| | | LSmeans (s.e.) | | | |
|---|---|---|---|---|---|
| Trait | Mean (s.e.) | 11 | 12 | 22 | P-value |
| ccw | 202.2 (1.07) | 204.9 (3.55)ec | 197.3 (1.77)f | 198.4 (1.84)d | 0.14 |
| dirtywt | 247.4 (1.41) | 248.9 (4.87)ca | 240.4 (2.57)d | 240.7 (2.71)b | 0.24 |
| dripprct | 2.16 (0.13) | 2.23 (0.49) | 1.99 (0.24)a | 2.36 (0.27)b | 0.38 |
| firmness | 2.61 (0.11) | 3.39 (0.30)c | 2.78 (0.12)d | 2.71 (0.16)d | 0.14 |
| gcaloc_f | 12.93 (0.23) | 13.31 (0.76)a | 13.07 (0.45)c | 12.30 (0.46)bd | 0.19 |
| gcdays | 164.4 (0.76) | 158.3 (2.55)a | 162.2 (1.40)b | 162.3 (1.51)b | 0.30 |
| gcendwt | 113.5 (0.49) | 113.4 (1.56)ac | 111.3 (0.88)b | 110.4 (0.90)d | 0.23 |
| gcldg | 669.3 (2.87) | 675.4 (8.51)a | 663.7 (4.67)b | 661.0 (4.76)b | 0.30 |
| gctdg | 879.1 (4.56) | 885.5 (15.0)a | 866.5 (8.13)b | 862.8 (8.25)b | 0.39 |
| gcus_md | 67.14 (0.47) | 67.36 (1.32)ea | 64.42 (0.76)f | 65.17 (0.78)b | 0.10 |
| h_binwt | 25.57 (0.19) | 26.52 (0.48)ce | 25.67 (0.21)d | 25.16 (0.25)cf | 0.02 |
| h_blswt | 5.19 (0.04) | 5.39 (0.15)a | 5.20 (0.07)b | 5.12 (0.08)b | 0.23 |
| hammina | 8.64 (0.14) | 9.25 (0.53)a | 8.54 (0.21)b | 8.74 (0.25) | 0.42 |
| hamminb | 4.09 (0.15) | 5.19 (0.44)c | 4.36 (0.18)d | 4.29 (0.21)d | 0.18 |
| hamminl | 47.42 (0.44) | 49.11 (1.68) | 47.84 (0.75) | 47.69 (0.85) | 0.73 |
| hampH | 5.68 (0.01) | 5.62 (0.05)a | 5.69 (0.02)b | 5.68 (0.03)b | 0.34 |
| hcw | 204.1 (1.05) | 205.2 (3.43)ea | 197.6 (1.85)f | 199.1 (1.91)b | 0.13 |
| hprofat | 13.54 (0.19) | 13.00 (0.63) | 13.14 (0.34) | 13.14 (0.35) | 0.98 |
| hpromeat | 62.67 (0.79) | 63.27 (1.95)a | 59.89 (1.03)b | 60.28 (1.04)b | 0.29 |
| hprorib | 14.86 (0.55) | 8.88 (2.23)ge | 15.91 (0.86)h | 14.99 (1.06)f | 0.02 |
| japcs | 3.25 (0.06) | 2.98 (0.19)ec | 3.47 (0.10)f | 3.35 (0.10)d | 0.08 |
| l_binwt | 22.53 (0.19) | 23.17 (0.50)e | 21.91 (0.22)f | 22.67 (0.26)e | 0.01 |
| l_blswt | 8.13 (0.08) | 8.43 (0.23)e | 7.94 (0.10)fc | 8.19 (0.12)d | 0.06 |
| LMprct | 47.43 (0.15) | 48.16 (0.58)a | 47.36 (0.25)b | 47.69 (0.32) | 0.34 |
| loinmina | 6.69 (0.09) | 7.20 (0.32)a | 6.83 (0.18)b | 6.84 (0.19)b | 0.52 |
| loinminb | 2.94 (0.07) | 3.09 (0.18) | 3.03 (0.10) | 3.09 (0.10) | 0.86 |
| loinminl | 45.27 (0.19) | 44.86 (0.64) | 45.16 (0.35) | 45.19 (0.36) | 0.89 |
| loinpH | 5.73 (0.01) | 5.72 (0.03) | 5.73 (0.02) | 5.73 (0.02) | 0.95 |
| marbling | 2.21 (0.07) | 2.22 (0.20) | 2.37 (0.10) | 2.27 (0.11) | 0.64 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 12
Analysis of GNAS effect on meat quality and production traits in
commercial Landrace and Synthetic populations.

| | | LSmeans (s.e.) | | | |
|---|---|---|---|---|---|
| Trait | Mean (s.e.) | 11 | 12 | 22 | P-value |
| dirtywt | 245.5 (0.72) | 243.5 (1.84)a | 244.4 (1.38) | 245.9 (1.80)b | 0.53 |
| hcw | 198.0 (0.59) | 197.6 (1.47)a | 197.9 (1.11)a | 199.7 (1.42)bb | 0.40 |
| ccw | 195.8 (0.61) | 195.3 (1.49)a | 195.8 (1.11)a | 197.4 (1.44)bb | 0.46 |
| l_binwt | 21.34 (0.11) | 21.50 (0.23)a | 21.22 (0.17)bc | 21.65 (0.22)d | 0.11 |
| l_blswt | 7.45 (0.04) | 7.57 (0.09) | 7.55 (0.07) | 7.54 (0.09) | 0.95 |
| loinminl | 44.63 (0.11) | 44.80 (0.29)a | 44.40 (0.22)b | 44.59 (0.29) | 0.30 |
| loinmina | 6.69 (0.05) | 6.73 (0.14) | 6.74 (0.10) | 6.81 (0.13) | 0.87 |
| loinminb | 2.94 (0.04) | 3.17 (0.08) | 3.09 (0.06)a | 3.20 (0.08)b | 0.36 |
| japcs | 3.29 (0.03) | 3.24 (0.08)ea | 3.40 (0.06)f | 3.34 (0.07)b | 0.05 |
| marbling | 1.84 (0.03) | 1.92 (0.06) | 1.98 (0.05) | 1.96 (0.06) | 0.58 |
| firmness | 2.74 (0.06) | 3.20 (0.09)a | 3.07 (0.07)ba | 3.22 (0.09)b | 0.15 |
| loinpH | 5.70 (0.01) | 5.71 (0.01) | 5.71 (0.01) | 5.71 (0.01) | 0.72 |
| h_binwt | 23.61 (0.14) | 24.17 (0.24) | 24.19 (0.18)a | 23.90 (0.24)b | 0.50 |
| h_blswt | 4.58 (0.03) | 4.86 (0.05)aa | 4.79 (0.04)b | 4.77 (0.05)b | 0.25 |
| hamminl | 47.34 (0.20) | 48.23 (0.52)a | 48.31 (0.39)a | 47.47 (0.51)bb | 0.26 |
| hammina | 8.62 (0.08) | 8.79 (0.22) | 8.80 (0.16) | 8.58 (0.21) | 0.58 |
| hamminb | 4.28 (0.08) | 4.73 (0.18)a | 4.79 (0.13)c | 4.48 (0.18)bd | 0.23 |
| hampH | 5.67 (0.01) | 5.69 (0.02) | 5.69 (0.01) | 5.68 (0.02) | 0.69 |
| dripprct | 2.67 (0.08) | 2.43 (0.20) | 2.40 (0.14)a | 2.63 (0.19)b | 0.47 |
| hprofat | 13.10 (0.10) | 12.89 (0.27)aa | 13.25 (0.21)b | 13.26 (0.26)b | 0.30 |
| hpromeat | 55.89 (0.49) | 58.12 (0.78) | 58.43 (0.61) | 57.94 (0.76) | 0.77 |
| hprorib | 13.52 (0.23) | 12.65 (0.62)g | 14.13 (0.47)hc | 13.05 (0.63)d | 0.01 |
| LMprct | 46.93 (0.07) | 47.37 (0.20)aa | 47.18 (0.16)b | 47.11 (0.21)b | 0.39 |
| gcaloc_f | 12.94 (0.13) | 12.94 (0.33)e | 12.87 (0.25)e | 12.10 (0.32)ff | 0.04 |
| gcendwt | 113.0 (0.27) | 112.7 (0.70)a | 112.4 (0.54) | 111.8 (0.69)b | 0.52 |
| gcdays | 161.0 (0.53) | 154.3 (1.11)cc | 156.1 (0.85)d | 156.8 (1.09)d | 0.13 |
| gcldg | 672.5 (1.67) | 669.8 (3.91) | 668.7 (2.97) | 667.4 (3.84) | 0.89 |
| gctdg | 891.3 (2.89) | 890.0 (6.91) | 888.7 (5.22) | 887.4 (6.78) | 0.96 |
| gcus_md | 63.22 (0.32) | 62.33 (0.69) | 62.47 (0.52) | 62.12 (0.68) | 0.88 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 13
Analysis of MC3R effect on meat quality and production traits in
commercial Landrace population.

| | | LSmeans (s.e.) | | | |
|---|---|---|---|---|---|
| Trait | Mean (s.e.) | 11 | 12 | 22 | P-value |
| dirtywt | 244.9 (0.81) | 245.1 (1.76) | 246.3 (1.33) | 245.3 (1.66) | 0.78 |
| hcw | 194.9 (0.66) | 195.1 (1.46) | 195.9 (1.11) | 194.8 (1.36) | 0.74 |
| ccw | 192.7 (0.67) | 192.6 (1.49) | 193.4 (1.13) | 193.1 (1.41) | 0.90 |
| l_binwt | 20.92 (0.12) | 20.94 (0.25) | 20.81 (0.19) | 20.90 (0.23) | 0.85 |
| l_blswt | 7.22 (0.05) | 7.08 (0.09)a | 7.22 (0.08)b | 7.16 (0.09) | 0.32 |
| loinminl | 44.38 (0.14) | 44.06 (0.33) | 44.10 (0.26) | 44.32 (0.31) | 0.75 |
| loinmina | 6.69 (0.06) | 6.84 (0.14) | 6.73 (0.10) | 6.68 (0.13) | 0.64 |
| loinminb | 3.01 (0.05) | 3.11 (0.09) | 3.05 (0.07) | 3.09 (0.09) | 0.83 |
| japcs | 3.33 (0.03) | 3.47 (0.08)a | 3.35 (0.06)b | 3.40 (0.07) | 0.36 |
| marbling | 1.72 (0.03) | 1.68 (0.06)c | 1.67 (0.04)e | 1.82 (0.05)df | 0.04 |
| firmness | 2.74 (0.07) | 2.95 (0.09)a | 2.85 (0.07)b | 2.89 (0.08) | 0.55 |
| loinpH | 5.68 (0.01) | 5.69 (0.01) | 5.69 (0.01) | 5.69 (0.01) | 0.88 |
| h_binwt | 22.99 (0.15) | 22.68 (0.27)a | 22.89 (0.20)a | 23.20 (0.25)b | 0.31 |
| h_blswt | 4.38 (0.03) | 4.32 (0.05)e | 4.45 (0.04)fc | 4.36 (0.05)d | 0.02 |
| hamminl | 47.40 (0.22) | 47.71 (0.52) | 48.00 (0.41)a | 47.30 (0.49)b | 0.40 |
| hammina | 8.67 (0.10) | 8.72 (0.22) | 8.72 (0.16) | 8.93 (0.20) | 0.62 |
| hamminb | 4.44 (0.09) | 4.57 (0.20) | 4.54 (0.15) | 4.62 (0.19) | 0.93 |
| hampH | 5.66 (0.01) | 5.67 (0.02)a | 5.69 (0.01)b | 5.69 (0.01)b | 0.43 |
| dripprct | 2.78 (0.09) | 2.68 (0.20)a | 2.69 (0.15)a | 2.41 (0.18)b | 0.36 |
| hprofat | 12.96 (0.12) | 13.23 (0.27) | 13.10 (0.21) | 13.00 (0.25) | 0.78 |
| hpromeat | 54.96 (0.34) | 54.63 (0.81)a | 55.61 (0.65)b | 55.17 (0.76) | 0.46 |
| hprorib | 13.33 (0.25) | 12.39 (0.58)a | 13.24 (0.46)bc | 12.22 (0.56)d | 0.17 |
| LMprct | 46.78 (0.07) | 46.70 (0.18) | 46.88 (0.15) | 46.92 (0.17) | 0.55 |
| gcaloc_f | 12.87 (0.15) | 12.53 (0.37) | 12.75 (0.29) | 12.78 (0.34) | 0.83 |
| gcendwt | 112.6 (0.33) | 111.6 (0.74)c | 112.9 (0.57)d | 112.5 (0.70) | 0.22 |
| gcdays | 158.2 (0.74) | 157.9 (1.05)a | 156.3 (0.81)b | 157.7 (1.20)a | 0.32 |
| gcldg | 675.0 (2.10) | 658.1 (4.65)ea | 668.4 (3.57)f | 665.1 (4.04)b | 0.11 |
| gctdg | 902.5 (3.67) | 885.4 (8.23)ca | 902.3 (6.28)d | 896.2 (7.15)b | 0.16 |
| gcus_md | 60.43 (0.39) | 60.71 (0.87) | 60.51 (0.64) | 59.91 (0.72) | 0.67 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

TABLE 14
Analysis of MC3R effect on meat quality and production traits in
commercial Synthetic population.

| | | LSmeans (s.e.) | | | |
|---|---|---|---|---|---|
| Trait | Mean (s.e.) | 11 | 12 | 22 | P-value |
| dirtywt | 248.5 (1.45) | 243.2 (2.61) | 244.1 (3.51) | 244.4 (19.9) | 0.97 |
| hcw | 204.5 (1.06) | 201.8 (1.72) | 200.8 (2.63) | 199.9 (17.3) | 0.94 |
| ccw | 202.7 (1.09) | 200.9 (1.73) | 200.9 (2.60) | 195.9 (17.3) | 0.96 |
| l_binwt | 22.46 (0.19) | 21.96 (0.24) | 22.04 (0.31) | 22.35 (1.57) | 0.96 |
| l_blswt | 8.13 (0.08) | 7.86 (0.11)ea | 8.26 (0.14)f | 8.66 (0.69)b | 0.06 |
| loinminl | 45.17 (0.19) | 45.20 (0.33) | 44.72 (0.49) | 43.01 (3.03) | 0.56 |
| loinmina | 6.67 (0.09) | 6.73 (0.18)a | 7.08 (0.25)b | 6.55 (1.39) | 0.38 |
| loinminb | 3.03 (0.07) | 3.35 (0.09) | 3.29 (0.13) | 3.70 (0.86) | 0.83 |
| japcs | 3.30 (0.05) | 3.41 (0.10) | 3.42 (0.13) | 2.95 (0.67) | 0.79 |
| marbling | 2.22 (0.07) | 2.25 (0.09) | 2.23 (0.13) | 2.34 (0.70) | 0.98 |
| firmness | 2.81 (0.11) | 3.07 (0.12) | 3.21 (0.14) | 3.31 (0.64) | 0.69 |
| loinpH | 5.73 (0.01) | 5.73 (0.02) | 5.74 (0.02)a | 5.57 (0.15)b | 0.51 |
| h_binwt | 25.65 (0.20) | 25.64 (0.23) | 25.92 (0.30) | 25.83 (1.43) | 0.74 |
| h_blswt | 5.19 (0.04) | 5.15 (0.07) | 5.21 (0.09) | 5.41 (0.43) | 0.77 |
| hamminl | 47.41 (0.43) | 48.23 (0.77) | 48.05 (1.00) | 46.93 (4.48) | 0.95 |
| hammina | 8.52 (0.13) | 8.82 (0.25) | 8.44 (0.33) | 9.46 (1.41) | 0.48 |
| hamminb | 4.27 (0.15) | 5.08 (0.19) | 4.84 (0.25) | 5.11 (1.24) | 0.73 |
| hampH | 5.68 (0.01) | 5.68 (0.03) | 5.66 (0.04) | 5.56 (0.13) | 0.65 |
| dripprct | 2.13 (0.12) | 2.05 (0.23) | 2.26 (0.29) | 2.55 (1.18) | 0.77 |
| hprofat | 13.69 (0.19) | 13.73 (0.32) | 13.49 (0.48) | 10.82 (2.88) | 0.57 |
| hpromeat | 63.65 (0.52) | 62.66 (0.88)a | 64.80 (1.35)b | 62.68 (8.23) | 0.35 |
| hprorib | 14.49 (0.52) | 13.92 (0.94)a | 14.47 (1.08)a | 6.01 (5.02)b | 0.25 |
| LMprct | 47.25 (0.16) | 47.00 (0.26) | 47.33 (0.30) | 48.04 (1.34) | 0.56 |
| gcaloc_f | 12.83 (0.23) | 12.68 (0.42) | 13.21 (0.59) | 12.79 (3.37) | 0.69 |
| gcendwt | 113.7 (0.50) | 112.5 (0.81)c | 110.1 (1.24)d | 114.0 (8.40) | 0.20 |
| gcdays | 163.1 (0.84) | 157.2 (1.38)c | 161.5 (1.98)d | 153.7 (10.6) | 0.14 |
| gcldg | 673.0 (3.15) | 667.0 (4.33)a | 655.7 (6.88)b | 698.1 (43.1) | 0.23 |
| gctdg | 878.3 (4.71) | 870.3 (7.23)a | 853.3 (11.9)b | 905.9 (77.6) | 0.38 |
| gcus_md | 66.57 (0.48) | 64.24 (0.71) | 63.16 (1.01) | 64.13 (5.94) | 0.60 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

Table 14 indicates the effect of MC3R genotypes on several traits in a commercial Synthetic population. As only one animal carrying MC3R genotype was detected in this population, the comparison is essentially made between MC3R genotypes 11 and 12.

TABLE 15
Overall analysis of MC3R effect on meat quality and production traits in
commercial Landrace and Synthetic populations

| | | LSmeans (s.e.) | | | |
|---|---|---|---|---|---|
| Trait | Mean (s.e.) | 11 | 12 | 22 | P-value |
| dirtywt | 246.0 (0.72) | 245.3 (1.46) | 246.1 (1.52) | 245.0 (2.01) | 0.81 |
| hcw | 198.2 (0.59) | 199.1 (1.14) | 199.2 (1.21) | 198.1 (1.61) | 0.78 |
| ccw | 196.0 (0.60) | 196.7 (1.15) | 196.9 (1.22) | 196.5 (1.63) | 0.96 |
| l_binwt | 21.35 (0.11) | 21.33 (0.19) | 21.25 (0.19) | 21.28 (0.24) | 0.92 |
| l_blswt | 7.47 (0.04) | 7.45 (0.08)ea | 7.63 (0.08)f | 7.57 (0.10)b | 0.10 |
| loinminl | 44.65 (0.11) | 44.55 (0.24) | 44.56 (0.25) | 44.79 (0.33) | 0.75 |
| loinmina | 6.68 (0.05) | 6.77 (0.11) | 6.75 (0.11) | 6.64 (0.15) | 0.70 |
| loinminb | 3.02 (0.04) | 3.24 (0.06) | 3.23 (0.07) | 3.25 (0.09) | 0.96 |
| japcs | 3.32 (0.03) | 3.44 (0.06)a | 3.33 (0.06)b | 3.37 (0.08) | 0.26 |
| marbling | 1.86 (0.03) | 1.96 (0.05)c | 1.95 (0.05)e | 2.09 (0.07)df | 0.09 |
| firmness | 2.75 (0.06) | 3.14 (0.07) | 3.08 (0.07) | 3.12 (0.09) | 0.74 |
| loinpH | 5.70 (0.01) | 5.71 (0.01) | 5.71 (0.01) | 5.71 (0.01) | 0.97 |
| h_binwt | 23.72 (0.14) | 24.00 (0.19)c | 24.17 (0.20)a | 24.48 (0.26)db | 0.25 |
| h_blswt | 4.60 (0.03) | 4.74 (0.04)e | 4.84 (0.05)fa | 4.76 (0.06)b | 0.08 |
| hamminl | 47.40 (0.20) | 47.96 (0.43) | 48.34 (0.44)a | 47.68 (0.57)b | 0.40 |
| hammina | 8.63 (0.08) | 8.65 (0.16) | 8.64 (0.17) | 8.83 (0.22) | 0.63 |
| hamminb | 4.39 (0.08) | 4.74 (0.15) | 4.76 (0.15) | 4.83 (0.20) | 0.91 |
| hampH | 5.66 (0.01) | 5.68 (0.01) | 5.68 (0.01) | 5.69 (0.02) | 0.89 |
| dripprct | 2.58 (0.07) | 2.39 (0.15) | 2.48 (0.15)a | 2.18 (0.20)b | 0.29 |
| hprofat | 13.19 (0.10) | 13.51 (0.21) | 13.37 (0.22) | 13.25 (0.29) | 0.72 |
| hpromeat | 57.72 (0.32) | 59.42 (0.61)c | 60.67 (0.64)d | 60.32 (0.83) | 0.20 |
| hprorib | 13.60 (0.23) | 13.24 (0.51)a | 13.96 (0.49)be | 12.66 (0.65)f | 0.09 |
| LMprct | 46.88 (0.07) | 46.91 (0.16)a | 47.13 (0.15)b | 47.19 (0.20)b | 0.30 |
| gcaloc_f | 12.86 (0.13) | 12.58 (0.27) | 12.86 (0.29) | 12.90 (0.37) | 0.64 |
| gcendwt | 113.0 (0.28) | 112.5 (0.58) | 112.9 (0.61) | 112.7 (0.81) | 0.86 |
| gcdays | 160.2 (0.57) | 155.3 (0.91) | 155.1 (0.96) | 156.0 (1.48) | 0.80 |
| gcldg | 674.3 (1.75) | 666.0 (3.22)a | 670.8 (3.42)b | 669.6 (4.37) | 0.49 |
| gctdg | 893.3 (2.93) | 886.5 (5.62) | 892.9 (5.99) | 890.7 (7.70) | 0.67 |
| gcus_md | 62.92 (0.33) | 61.47 (0.65) | 60.83 (0.64) | 60.55 (0.80) | 0.54 |

Significance levels used:
a, b - 0.3;
c, d - 0.1;
e, f - 0.05;
g, h - 0.01;
i, j - 0.005;
k, l - 0.001;
m, n - 0.0005;
o, p - 0.0001

The results determined in these commercial lines suggest strong associations with color related traits (loin and ham minolta scores) and other meat quality traits as well as with growth and fatness. These are all valuable traits for the pork industry. These markers may also be used together; in this strategy selection will be possible, not only for meat quality traits but also for growth and fatness traits.

### EXAMPLE 4

Several Quantitative Trait Loci (QTL) for meat quality traits were detected on swine chromosome 17 (SSC17), including color, lab loin hunter, lab loin minolta, average lactate and average glycolytic potential (Malek et al., 2001). See initial QTL FIG. 1. The inventors mapped three genes on the SSC17 QTL region: PKIG (protein kinase inhibitor gamma), PTPN1 (protein tyrosine phosphatase, non-receptor type 1) and PPP1R3D (protein phosphatase 1, regulatory subunit 3D). Following these results, three more genes were mapped in the same SSC17 QTL region: CTSZ (Cathepsin Z), GNAS (guanine nucleotide binding protein G (S), alpha subunit—adenylate cyclase stimulating G alpha protein) and MC3R (melanocortin-3 receptor).

Given the position in the SSC17 map of the above mentioned six genes, an effort was made to fine map this QTL region on SSC17. Using the available comparative maps between the human and pig genomes several positional candidate genes were chosen for study, in an attempt to find the gene(s) responsible for the observed phenotypic variation on SSC17.

A total of nine more genes were analyzed, namely MMP9 [matrix metalloproteinase 9 (gelatinase B, 92 kDa gelatinase, 92 kDa type IV collagenase)], ATP9A (ATPase, Class II, type 9A), CYP24A1 (cytochrome P450, family 24, subfamily A, polypeptide 1), AURKA (aurora kinase A), DOK5 (docling protein 5), RAE1 [RAE1 RNA export 1 homolog (S. pombe)], SPO11 [SPO11 meiotic protein covalently bound to DSB-like (S. cerevisiae)], RAB22A (RAB22A, member RAS oncogene family) and PCK1 [phosphoenolpyruvate carboxykinase 1 (soluble)].

Given the map position of these genes, they were considered as good candidate genes to explain the variation detected in the SSC17 pork meat quality traits QTL. PCR-RFLP tests were developed for polymorphisms in these genes and used to map most of these genes underneath the SSC17 QTL peaks for color, lab loin hunter, lab loin Minolta, average lactate and average glycolytic potential. These QTL span the region on SSC17 that goes approximately from 70 to 107 cM.

The position of the genes on the map is as follows: PKIG maps to 70.4 cM, MMP9 to 72.6 cM, PTPN1 to 80.4 cM, ATP9A to 83.6 cM, CYP24A1 to 85.3 cM, DOK5 to 88.3 cM, MC3R to 88.3 cM, AURKA to 90.4 cM, SPO11 to 97.4 cM, RAE1 to 98.9 cM, RAB22A to 100.3 cM, GNAS to 102.5 cM, CTSZ to 103.4 cM and PPP1R3D to 107.5 cM. The map position of two genes (PCK1 and C20orf43) has not yet been determined. PCK1 is expected to map between RAE1 and RAB22A. The effect on several economic traits of the variants of all sixteen genes analyzed were investigated in the Iowa State University Berkshire×Yorkshire cross (Table 16).

TABLE 16
Results of the associations with several growth, carcass composition and meat quality traits in an ISU pig resource
population

| | PKIG | MMP9 | PTPN1 | ATP9A | CYP24A1 | DOK5 | MC3R | AURKA | SPO11 | RAE1 | PCK1 |
|---|---|---|---|---|---|---|---|---|---|---|---|
| Ave. backfat | 0.2111 | 0.0171 | 0.2122 | 0.1279 | [symbol] | [symbol] | [symbol] | [symbol] | [symbol] | [symbol] | 0.2706 |
| Carcass wt. | 0.8243 | 0.5551 | 0.3084 | 0.7018 | 0.7148 | 0.5038 | [symbol] | [symbol] | 0.3126 | 0.3317 | 0.0252 |
| Cholesterol | 0.8197 | 0.9055 | 0.8768 | 0.8607 | 0.0518 | 0.2322 | 0.5107 | 0.584 | 0.9165 | 0.9878 | 0.9638 |
| Last rib backfat | 0.321 | 0.0046 | 0.3322 | 0.1566 | 0.1175 | 0.0577 | 0.3568 | 0.1378 | 0.1634 | 0.1155 | 0.2199 |
| Loin eye area | 0.479 | 0.1722 | 0.1749 | 0.6434 | 0.3872 | 0.5588 | [symbol] | [symbol] | 0.3391 | 0.3223 | 0.4353 |
| Length | 0.0454 | 0.2806 | 0.1294 | [symbol] | [symbol] | 0.1048 | 0.7108 | 0.8736 | 0.1034 | 0.2249 | 0.0971 |
| Lumbar backfat | 0.3446 | 0.0537 | 0.4087 | [symbol] | [symbol] | [symbol] | [symbol] | [symbol] | 0.0957 | 0.1429 | 0.2463 |
| Marbling score | 0.3508 | [symbol] | [symbol] | 0.1006 | 0.7645 | 0.0221 | 0.7956 | 0.912 | 0.6907 | 0.8043 | 0.7126 |
| Tenth rib backfat | 0.1929 | 0.0452 | 0.2011 | 0.6511 | 0.1108 | 0.1548 | 0.0511 | 0.012 | 0.1682 | 0.1486 | 0.7022 |
| Total lipid perc. | 0.0869 | 0.3125 | 0.7891 | 0.3109 | 0.3081 | 0.087 | 0.9587 | 0.615 | 0.5485 | 0.6163 | 0.7122 |
| Ave. daily gain | [symbol] | [symbol] | 0.2511 | 0.5607 | 0.0853 | 0.8057 | 0.7461 | 0.868 | 0.2441 | 0.5103 | 0.8671 |
| Daily gain test | 0.8494 | 0.4577 | 0.1485 | 0.1801 | 0.037 | 0.25 | [symbol] | [symbol] | 0.6122 | 0.9286 | 0.8214 |
| Ave. drip | 0.4317 | 0.3022 | [symbol] | [symbol] | [symbol] | [symbol] | 0.7524 | 0.898 | 0.427 | 0.2356 | 0.3645 |
| Birth wt. | 0.7328 | 0.3094 | 0.1691 | 0.8458 | 0.7723 | 0.3449 | [symbol] | [symbol] | 0.1855 | 0.32 | 0.1789 |
| Color | 0.1028 | 0.4384 | 0.4277 | [symbol] | [symbol] | [symbol] | 0.4415 | 0.2338 | 0.1335 | 0.1481 | 0.1014 |
| Firmness score | 0.6369 | 0.8726 | 0.2 | 0.2449 | 0.8777 | 0.1702 | 0.426 | 0.6876 | 0.9797 | 0.8247 | 0.8221 |
| Fiber type I | 0.3143 | 0.8995 | 0.3465 | 0.1569 | 0.3519 | 0.0455 | 0.9595 | 0.9568 | 0.6975 | 0.3557 | 0.6124 |
| Fiber type II ratio | 0.1832 | 0.0265 | 0.8432 | 0.9583 | 0.3594 | 0.4061 | [symbol] | [symbol] | 0.5204 | 0.6035 | 0.4669 |
| Ham hunter | 0.6992 | 0.2932 | 0.9792 | 0.831 | 0.2075 | 0.6505 | 0.518 | 0.6689 | [symbol] | [symbol] | 0.4592 |
| Ham minolta | 0.4596 | 0.1567 | 0.9985 | 0.8223 | 0.1658 | 0.481 | 0.5497 | 0.7318 | [symbol] | [symbol] | 0.5998 |
| Ham pH | 0.474 | 0.8325 | 0.8837 | 0.5111 | 0.9265 | 0.0644 | 0.8317 | 0.7792 | 0.1909 | 0.0203 | 0.2841 |
| Horm loin hunter | 0.4859 | 0.5177 | 0.0893 | 0.5586 | 0.1635 | 0.8097 | 0.8303 | 0.6833 | 0.2213 | 0.2141 | 0.3089 |
| Horm loin minolta | 0.1753 | 0.7414 | 0.341 | 0.3322 | 0.3984 | 0.0208 | 0.3765 | 0.7036 | 0.7 | 0.9217 | 0.2027 |
| Horm loin min. pH | 0.4426 | 0.241 | 0.7292 | 0.137 | 0.4714 | 0.1404 | 0.3898 | 0.7525 | 0.3943 | 0.2321 | 0.6744 |
| Lab loin hunter | 0.1414 | 0.1913 | 0.5686 | [symbol] | [symbol] | [symbol] | 0.4971 | 0.8216 | 0.1385 | 0.2398 | 0.2019 |
| Lab loin minolta | 0.115 | 0.2036 | 0.5364 | [symbol] | [symbol] | [symbol] | 0.5311 | 0.8726 | 0.1185 | 0.207 | 0.1823 |
| Lab loin pH | 0.8086 | 0.874 | 0.836 | 0.1524 | 0.2711 | 0.3596 | 0.8869 | 0.8904 | 0.719 | 0.6875 | 0.8944 |
| Weaning wt. | 0.1102 | 0.0679 | 0.1871 | 0.5732 | 0.0811 | 0.7177 | 0.4003 | 0.694 | 0.2718 | 0.5334 | 0.9554 |
| Water holding ca. | 0.0555 | 0.6584 | 0.1967 | 0.1017 | 0.2445 | 0.5639 | 0.3261 | 0.5959 | 0.7244 | 0.8413 | 0.7853 |
| Ave. instron force | 0.1496 | 0.8037 | 0.5758 | 0.3433 | 0.3247 | 0.4511 | 0.5945 | 0.4172 | 0.2529 | 0.2393 | 0.851 |
| Chew source | 0.9039 | 0.367 | 0.457 | 0.4152 | 0.1367 | 0.2807 | 0.7436 | 0.6801 | 0.532 | 0.4024 | 0.8294 |
| Flavor score | 0.6111 | 0.5987 | 0.5793 | 0.0143 | 0.5194 | 0.0116 | 0.1383 | 0.1849 | 0.7288 | 0.6746 | 0.3857 |
| Juciness score | 0.6949 | 0.9672 | 0.3216 | 0.0246 | 0.3035 | 0.758 | 0.3364 | 0.4277 | 0.8433 | 0.7165 | 0.7232 |
| Off flavor score | 0.4235 | 0.6999 | 0.653 | 0.0945 | 0.6348 | 0.0136 | 0.1119 | 0.3564 | 0.6389 | 0.4124 | 0.7374 |
| Cooking loss | 0.919 | 0.8192 | 0.5624 | 0.3491 | 0.172 | 0.6246 | 0.817 | 0.6831 | [symbol] | [symbol] | [symbol] |
| Tenderness | 0.5385 | 0.8673 | 0.4155 | 0.1779 | 0.2619 | 0.3811 | 0.8192 | 0.6919 | 0.1468 | 0.0869 | 0.5639 |
| Ave. glycogen | 0.6995 | 0.8379 | 0.2134 | 0.5389 | 0.2884 | 0.3759 | 0.0688 | 0.1753 | 0.775 | 0.6295 | 0.3844 |
| Ave. glycolytic pt. | 0.8384 | 0.9815 | [symbol] | [symbol] | 0.1342 | 0.3624 | 0.0379 | 0.1213 | 0.824 | 0.573 | 0.9926 |
| Ave. lactate | 0.925 | 0.9362 | [symbol] | [symbol] | 0.1303 | 0.1311 | 0.0887 | 0.2078 | 0.5795 | 0.2609 | 0.7906 |

| | RAB22A | GNAS | CTSZ | PPP1R3D |
|---|---|---|---|---|
| Ave. backfat | 0.212 | 0.9694 | 0.2462 | 0.1059 |
| Carcass wt. | 0.7802 | 0.7506 | 0.3839 | 0.0968 |
| Cholesterol | 0.8952 | 0.7917 | 0.7515 | 0.5105 |
| Last rib backfat | 0.4736 | 0.9713 | 0.481 | 0.1851 |
| Loin eye area | 0.8051 | 0.8501 | 0.4606 | 0.0087 |
| Length | 0.1906 | 0.7328 | 0.3637 | 0.9074 |
| Lumbar backfat | 0.0649 | 0.7376 | [symbol] | [symbol] |
| Marbling score | 0.9821 | 0.6636 | 0.7897 | 0.1714 |
| Tenth rib backfat | 0.6631 | 0.7501 | 0.4659 | 0.2744 |
| Total lipid perc. | 0.9087 | 0.2782 | 0.9773 | 0.5371 |
| Ave. daily gain | 0.008 | 0.5062 | 0.503 | 0.1219 |
| Daily gain test | 0.5476 | 0.6492 | 0.5692 | 0.0103 |
| Ave. drip | 0.4989 | [symbol] | [symbol] | 0.7416 |
| Birth wt. | 0.2773 | 0.6668 | 0.2643 | 0.0249 |
| Color | [symbol] | [symbol] | [symbol] | 0.7563 |
| Firmness score | 0.7006 | 0.4368 | 0.8751 | 0.8338 |
| Fiber type I | 0.8608 | 0.7141 | 0.5117 | 0.1068 |
| Fiber type II ratio | 0.3354 | 0.3416 | 0.9076 | 0.9058 |
| Ham hunter | 0.0463 | 0.7496 | 0.7693 | 0.2836 |
| Ham minolta | 0.0443 | 0.8835 | 0.7703 | 0.2757 |
| Ham pH | 0.3977 | 0.9717 | 0.7565 | 0.2582 |
| Horm loin hunter | 0.0358 | 0.2908 | 0.3707 | 0.5178 |
| Horm loin minolta | 0.569 | 0.2328 | 0.4654 | 0.0782 |
| Horm loin min. pH | 0.2462 | 0.5182 | 0.9979 | 0.8604 |
| Lab loin hunter | [symbol] | [symbol] | [symbol] | 0.9191 |
| Lab loin minolta | [symbol] | [symbol] | [symbol] | 0.9586 |
| Lab loin pH | 0.5542 | 0.8297 | 0.6126 | 0.904 |
| Weaning wt. | 0.0052 | 0.6199 | 0.3849 | 0.0651 |
| Water holding ca. | 0.586 | 0.0753 | 0.1775 | 0.4714 |
| Ave. instron force | 0.0958 | 0.1863 | 0.1792 | 0.5643 |
| Chew source | 0.1955 | 0.0312 | 0.1051 | 0.4717 |
| Flavor score | 0.1552 | 0.2907 | 0.0072 | 0.4142 |
| Juciness score | 0.1874 | 0.6349 | 0.0034 | 0.6381 |
| Off flavor score | 0.5313 | 0.755 | 0.1822 | 0.3466 |
| Cooking loss | [symbol] | 0.8986 | 0.0197 | 0.7415 |
| Tenderness | [symbol] | [symbol] | [symbol] | 0.9105 |
| Ave. glycogen | 0.31 | 0.5007 | 0.4841 | 0.4856 |
| Ave. glycolytic pt. | 0.9504 | 0.2944 | 0.2987 | 0.9866 |
| Ave. lactate | 0.7658 | 0.3163 | 0.3314 | 0.8646 |
| Significant effects (P < 0.1) are indicated in bold. Chromosonial regions associated with growth, fat and meat quality traits are, highlighted in orange, green and purple, respectively. Individual effects of each gene on several traits are highlighted in yellow. Fatness traits Ave backfat, cholesterol, last rib backfat, lumbar backfat, marbling score, tenth rib backfat; growth traits carcass weight, loin eye depth, length, ave. daily gain, daily gain test, | | | | |
| # birth weight, fiber type I, fiber type II ratio, and weaning weight and remaining traits are meat quality traits, | | | | |

The results indicate that strong associations exist between several genes and the QTL traits on SSC17. Moreover, additional and very significant effects on growth and fat traits were also detected, as well as several associations with other meat quality traits. PKIG and MMP9 showed associations mostly with fat and growth traits. This chromosomal region was significantly associated with average daily gain. In addition, PKIG also showed to have a significant effect on length. MMP9 significantly affected several backfat traits, including last rib, lumbar, tenth rib and average backfat, and also had an influence on marbling score.

When genes that map closer to the QTL peaks on SSC17 were analyzed, significant associations between some genes, namely the chromosomal region containing PTPN1-ATP9A-CYP24A1-DOK5, (80.4 cM-88.4 cM)and all the QTL traits were detected. In fact, the PTPN1-ATP9A chromosomal region (80.4 cM-83.6 cM) was shown to be significantly associated with average glycolytic potential and average lactate, while the region comprising ATP9A-CYP24A1-DOK5 (83.6 cM-88.3 cM) had a significant effect on color, lab loin hunter and lab loin Minolta. In addition, this interval also affected another important meat quality trait, namely average drip. Furthermore, the ATP9A-CYP24A1(83.6 cM-85.3 cM) region was also found to be associated with length (growth trait) and lumbar backfat (fat trait). ATP9A individually affected three more meat quality traits (flavor, off flavor and juiciness scores), while the CYP24A1 variants had a significant effect on average backfat, average daily gain and average daily gain on test.

Some of the genes that mapped underneath the QTL peaks did not show associations with all of the QTL traits. However, the region including CYP24A1-DOK5-MC3R-AURKA (85.3 cM-90.4) had a significant effect on average and lumbar backfat. In addition, DOK5 significantly influenced last rib backfat, marbling score and total lipid percentage, as well as other meat quality traits (ham pH, flavor and off flavor scores).

The chromosomal region MC3R-AURKA (88.3 cM-90.4 cM) had a very significant effect on several growth (carcass weight, loin eye area, average daily gain on test, birth weight, fiber type II ratio) and fat traits (average and lumbar backfat measurements). In addition, MC3R was also significantly associated with two QTL traits (average glycolytic potential and average lactate), as well as with a related trait (average glycogen content).

SPO11 and RAE1 were found to be associated not only with fat traits (average and lumbar backfat), but also with several meat quality traits (ham hunter, ham Minolta and cooking loss). PCK1 affected two growth traits (length and carcass weight) and one meat quality trait (cooking loss). This trait is significantly affected by the chromosomal region SPO11-RAE1-PCK1-RAB22A (97.4 cM-100.3 cM)

The chromosomal region containing RAB22A-GNAS-CTSZ (100.3 cM-103.4 cM) significantly affected some QTL traits (color, lab loin hunter, lab loin Minolta) and two other meat quality traits (average drip and tenderness score). In addition, RAB22A individually affected ham hunter, ham Minolta and average instron force, all meat quality traits. Furthermore, this gene had also a significant effect on growth (average daily gain, weaning weight) and fat (lumbar backfat) traits. GNAS and CTSZ individually affected several meat quality traits, including water holding capacity, cooking loss and chew, flavor and juiciness scores.

Lumbar backfat was significantly affected by the chromosomal region CTSZ- PPP1R3D (103.4 cM-107.5 cM). Finally, PPP1R3D was significantly associated with several growth traits (carcass weight, loin eye area, average daily gain on test, birth weight and weaning weight).

All these results indicate that these markers can be used in the selection of pigs with improved meat quality and growth traits.

In addition to the studies conducted in the ISU pig resource population, the effect of nine genes was also analyzed in several commercial pure and synthetic lines. The results are indicated on table 17.

TABLE 17
Association of PKIG, PTPN1, ATP9A, CYP24A1, MC3R, RABi,
RAB22A, GNAS and CTSZ genotypes with several growth, carcass
composition and meat quality traits in a commercial pig resource
population

| | PKI | PTPN | ATP9 | CYP24 | MG3 | RAE | RAB22 | GNA | CTS |
|---|---|---|---|---|---|---|---|---|---|
| | G | I | A | Al | R | I | A | S | Z |
| dirty wt | 0.89 | 0.4 | 0.02 | 0.79 | 0.81 | 0.52 | 0.4 | 0.53 | 0.51 |
| hcw | 0.87 | [symbol] | 0.15 | 0.73 | 0.78 | 0.55 | 0.5 | 0.40 | 0.86 |
| ccw | 0.56 | [symbol] | 0.31 | 0.9 | 0.96 | 0.82 | 0.63 | 0.46 | 0.84 |
| L_binwt | [symbol] | 0.41 | 0.41 | 0.37 | 0.92 | 0.65 | 0.44 | 0.11 | 0.36 |
| L_blswt | [symbol] | 0.44 | 0.25 | 0.48 | 0.1 | 0.6 | 0.11 | 0.95 | 0.81 |
| loinminl | [symbol] | 0.62 | 0.45 | 0.3 | 0.75 | 0.16 | 0.43 | 0.30 | 0.34 |
| loinmin a | [symbol] | 0.93 | 0.98 | [symbol] | 0.7 | 0.46 | 0.43 | 0.87 | 0.69 |
| loinmin b | 0.77 | 0.92 | 0.59 | 0.69 | 0.96 | 0.91 | 0.4 | 0.36 | 0.4 |
| japcs | [symbol] | [symbol] | 0.8 | 0.56 | 0.26 | 0.41 | 0.23 | [symbol] | 0.22 |
| marbling | [symbol] | 0.82 | 0.63 | 0.36 | [symbol] | 0.3 | 0.8 | 0.58 | 0.26 |
| firmness | 0.11 | 0.94 | [symbol] | 0.14 | 0.74 | [symbol] | 0.7 | 0.15 | 0.61 |
| loinpH | 0.34 | [symbol] | 0.64 | 0.39 | 0.97 | 0.54 | 0.17 | 0.72 | 0.96 |
| h_binwt | 0.45 | 0.93 | 0.5 | 0.81 | 0.25 | 0.24 | 0.37 | 0.50 | 0.5 |
| h_blswt | 0.66 | 0.15 | 0.95 | 0.25 | [symbol] | 0.48 | 0.75 | 0.25 | 0.57 |
| hammin I | 0.69 | 0.28 | 0.24 | 0.45 | 0.4 | 0.21 | 0.25 | 0.26 | [symbol] |
| hammin a | 0.6 | 0.71 | 0.66 | 0.34 | 0.63 | 0.54 | 0.82 | 0.58 | 0.81 |
| hammin b | 0.91 | 0.99 | 0.66 | 0.64 | 0.91 | 0.45 | 0.38 | 0.23 | 0.12 |
| hampH | 0.74 | 0.44 | 0.65 | 0.17 | 0.89 | 0.83 | 0.18 | 0.69 | 0.76 |
| dripprct | 0.95 | 0.15 | 0.38 | 0.15 | 0.29 | 0.63 | 0.18 | 0.47 | 0.98 |
| hprofat | 0.18 | 0.29 | 0.4 | 0.2 | 0.72 | 0.54 | 0.28 | 0.30 | 0.5 |
| hpromeat | 0.14 | 0.58 | 0.47 | 0.13 | 0.2 | 0.9 | [symbol] | 0.77 | 0.6 |
| hprorib | 0.99 | 0.44 | 0.41 | 0.98 | [symbol] | 0.12 | 0.3 | [symbol] | 0.76 |
| LM prct | 0.44 | 0.57 | 0.86 | 0.38 | 0.3 | 0.32 | 0.51 | 0.39 | 0.75 |
| aloc_f | 0.46 | 0.16 | 0.84 | 0.31 | 0.64 | 0.75 | [symbol] | [symbol] | [symbol] |
| endwt | 0.95 | 0.91 | 0.52 | 0.74 | 0.86 | 0.3 | 0.54 | 0.52 | [symbol] |
| days | 0.91 | 0.59 | 0.63 | 0.71 | 0.8 | 0.94 | 0.94 | 0.13 | [symbol] |
| LDG, | 0.95 | 0.63 | 0.45 | 0.91 | 0.49 | 0.38 | 0.36 | 0.89 | [symbol] |
| gld | | | | | | | | | |
| TDG, | 0.94 | 0.67 | 0.25 | 0.44 | 0.67 | 0.2 | 0.57 | 0.96 | [symbol] |
| gld | | | | | | | | | |
| US_MD | 0.85 | 0.68 | 0.77 | 0.82 | 0.54 | 0.47 | 0.37 | 0.88 | 0.6 |

Significant effects (P < 0.1) are indicated in bold. Chromosomal regions associated with growth, fat and meat quality traits are highlighted in orange, green and purple, respectively.

The results determined in these commercial lines suggest strong associations with color related traits (loin and ham minolta scores) and other meat quality traits as well as with growth and fatness. These are all valuable traits for the pork industry. We strongly believe that the best way that the industry can apply this information is to simultaneously use all of these genes as genetic markers. If this strategy is adopted, then it is very likely that selection will be possible not only for meat quality Traits but also for growth and fatness traits. Specifically, PKIG, MMP9, ATP9A, CYP24A1, DOK5, MC3R, AURKA, PCK1, RAB22A, GNAS, CTSZ and PPP1R3D can be used as markers to select for improved growth related traits. In addition, PKIG, MMP9, PTPN1, ATP9A, CYP24A1, DOK5, MC3R, AURKA, SPO11, RAE1, RAB22A, GNAS, CTSZ and PPP1R3D can be used as markers to select for improved fat related traits. Finally, PKIG, PTPN1, ATP9A, CYP24A1, DOK5, MC3R, SPO11, RAE1, PCK1, RAB22A, GNAS, CTSZ and PPP1R3D can be used as markers to select for improved meat quality traits. Therefore, the use of the genes mapped to the meat quality QTL region of SSC17 as genetic markers, either singly or in combination, to assist in the selection for improved growth, fatness and meat quality measures is warranted.

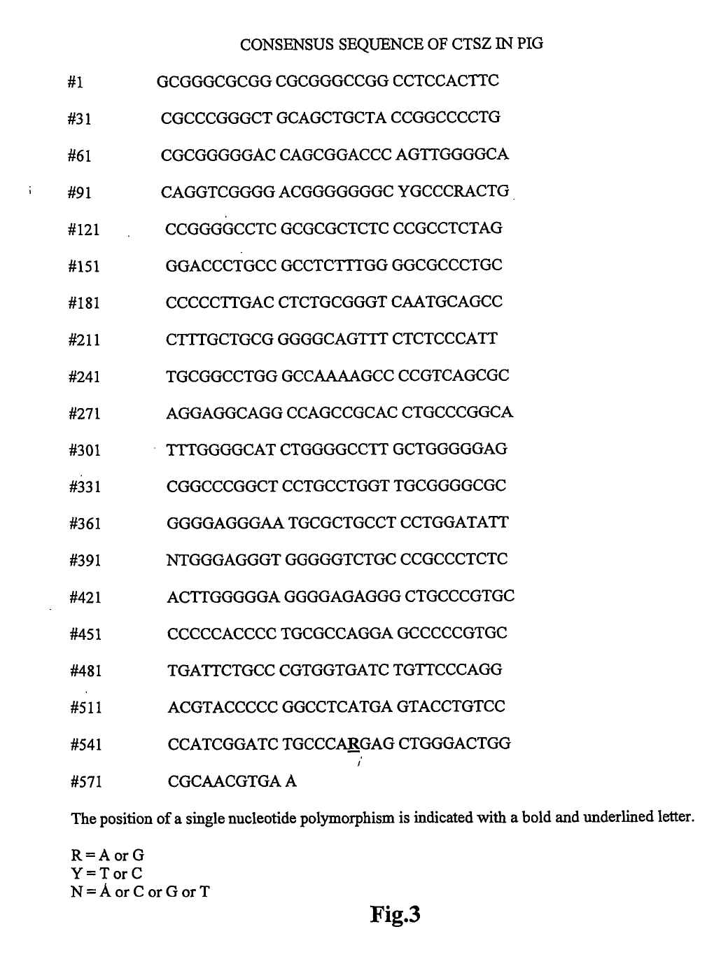

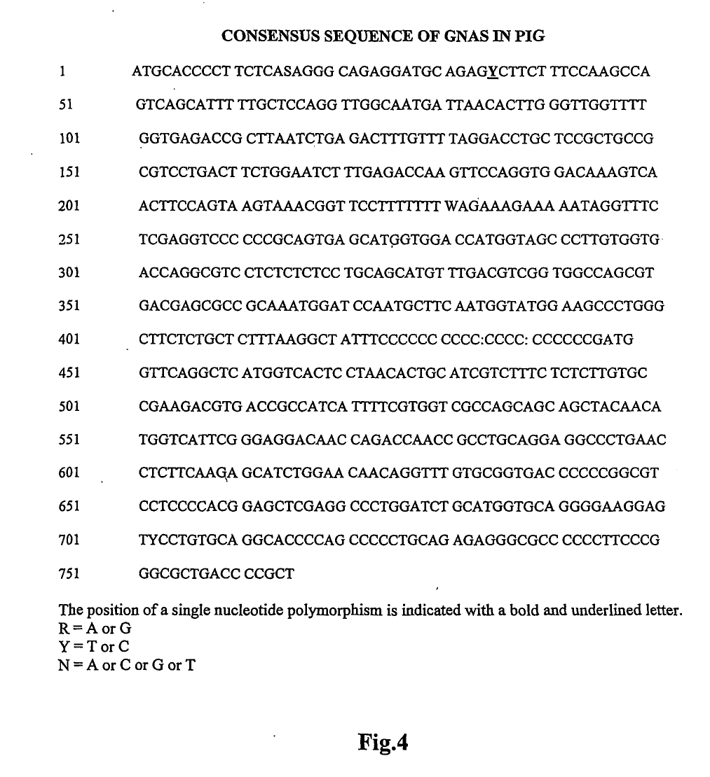

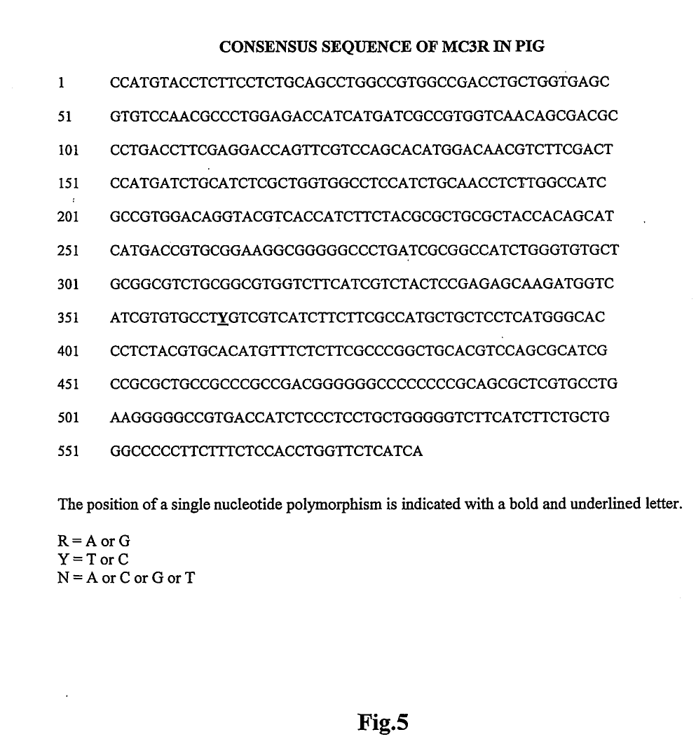

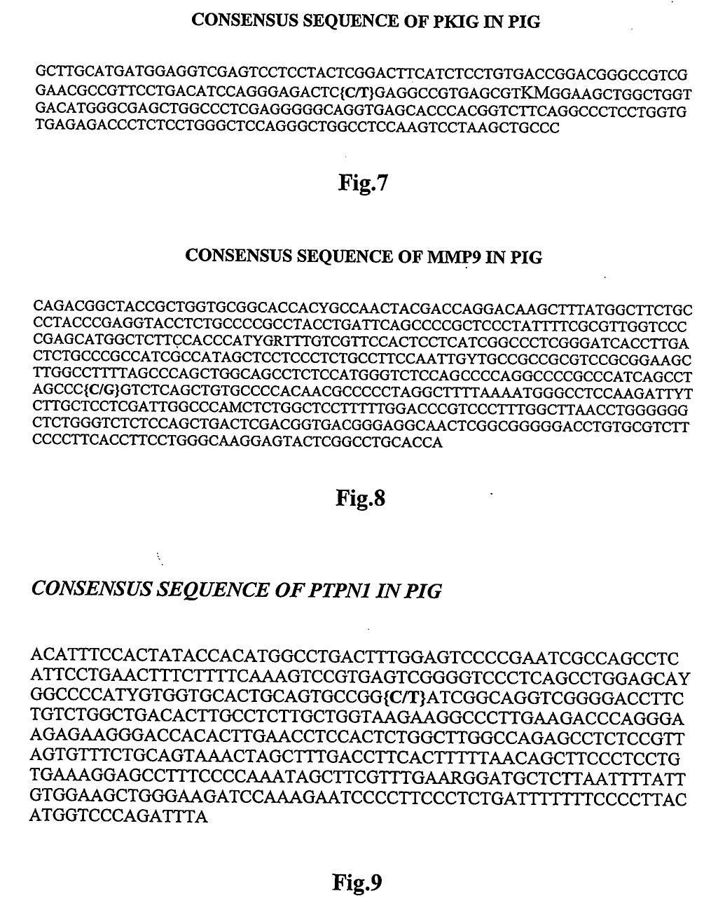

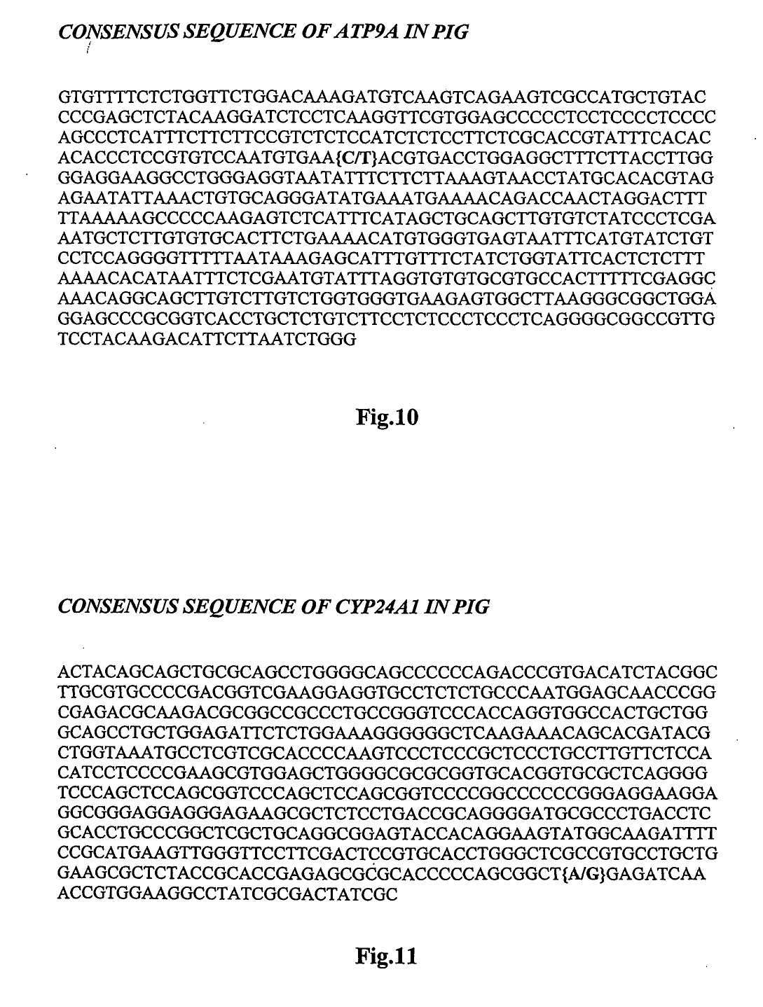

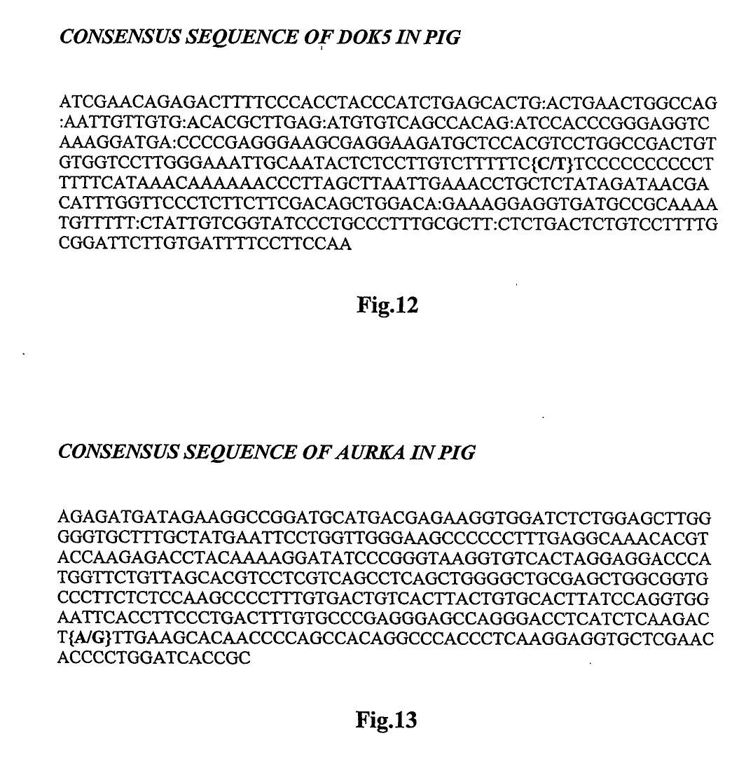

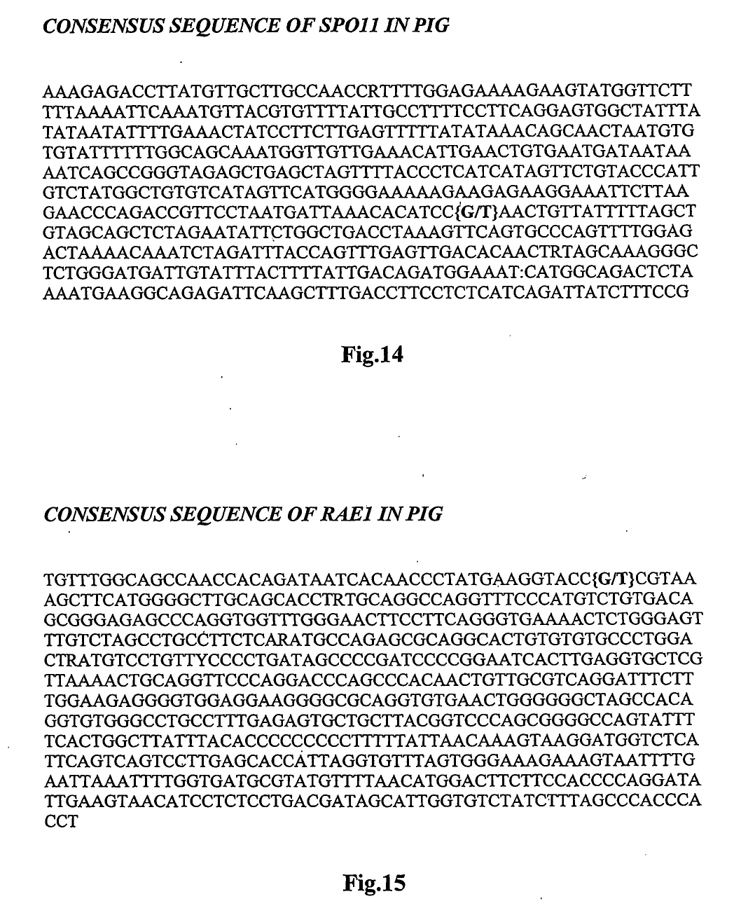

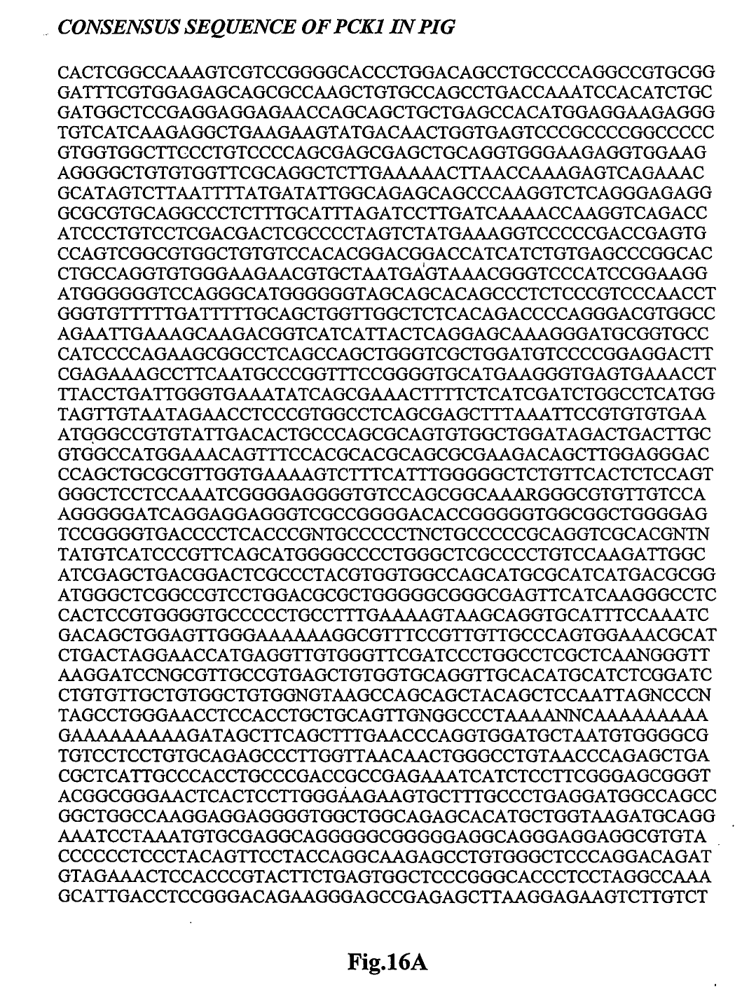

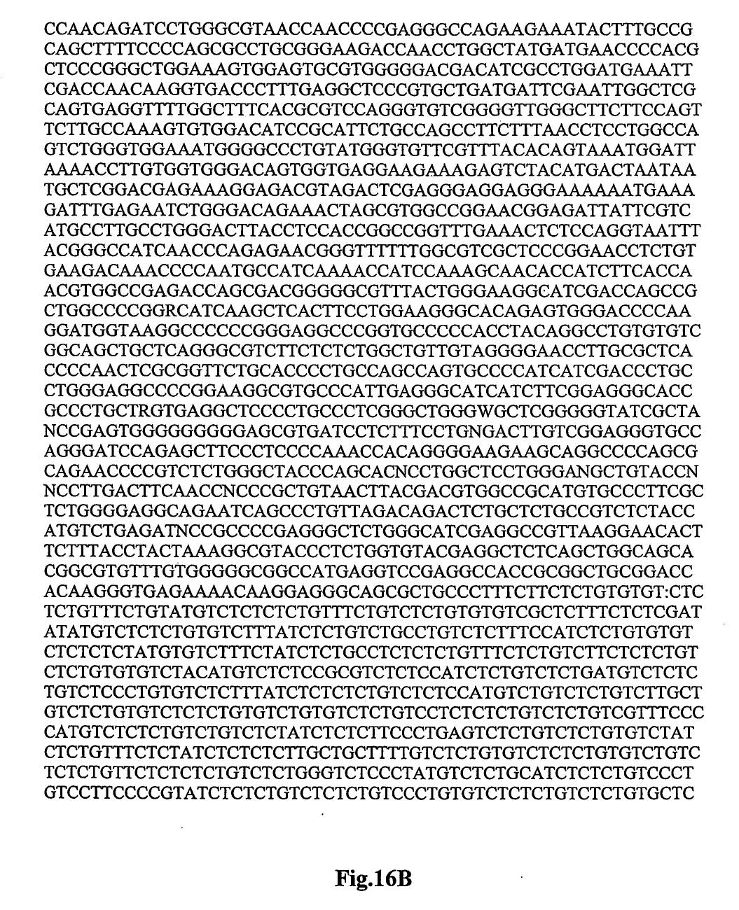

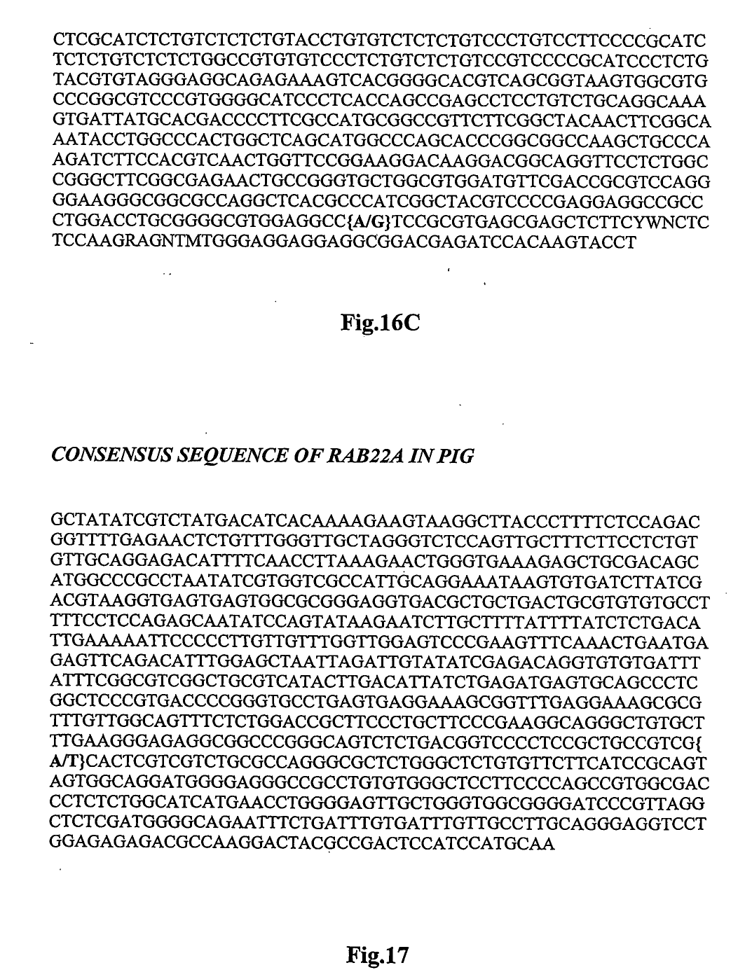

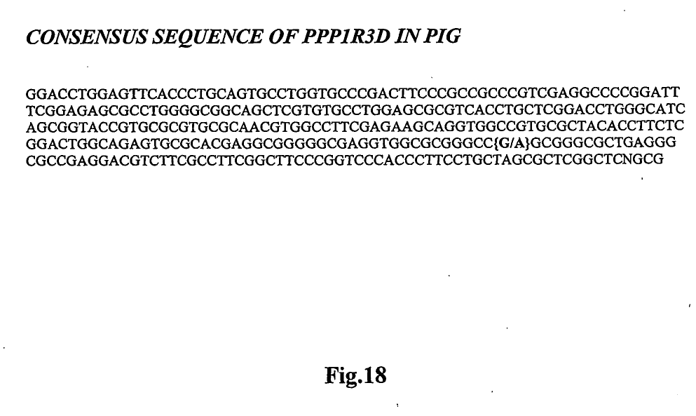

All PCR tests were performed using conditions listed earlier. The following is a list of primers, base changes and restriction enzymes used to generate the data earlier. All data is reported for the cut allele. Sequences of regions amplified by the primers including the base change are shown in FIGS. 3-5 and 7-18.

| | | | Base |
|---|---|---|---|
| Gene | Primer Sequences | Enzyme | Change |
| PKIG | F: 5′-GCTTGCATGATGGAGGTC-3′ | Dde I | C/T |
| | R: 5′-GGGCAGCTTAGGACTTGG-3′ | | |
| MMP9 | F: 5′-AGCCCCGCTCCCTATTTT-3′ | Msp I | C/G |
| | R: 5′-GAGTTGCCTCCCGTCACC-3′ | | |
| PTPN1 | F: 5′-ACATTTCCACTATACCACA-3′ | Nae I | C/T |
| | R: 5′-TAAATCTGGGACCATGTAA-3′ | | |
| ATP9A | F: 5′-TGGTTCTGGACAAAGATGTCA-3′ | Afl III | C/T |
| | R: 5′-ACACAAGAGCATTTCGAGGG-3′ | | |
| CYP24A1 | F: 5′-ACGATACGCTGGTAAATGCC-3′ | Alw NI | A/G |
| | R: 5′-CATAGCCCTCCTTGCGATAG-3′ | | |
| DOK5 | F: 5′-AACAGAGACTTTTCCCCCCTA-3′ | Bse RI | C/T |
| | R: 5′-GTTTTTTGTTTATGAAAGAGG-3′ | | |
| AURKA | F: 5′-AGATGATAGAAGGCCGGATG-3′ | Taa I | A/G |
| | R: 5′-GTGATCCAGGGGTGTTCG-3′ | | |
| SPO11 | F: 5′-AACCCAGACCGTTCCTAATG-3′ | Mse I | G/T |
| | R: 5′-GATAATCTGATGAGAGGAAGGTCAA-3′ | | |
| RAE1 | F: 5′-GGCAGCCAACCACAGATAA-3′ | Bst UI | G/T |
| | R: 5′-GGACCGTAAGCAGCACTCTC-3′ | | |
| PCK1 | F: 5′-GGCACGTCAGCGGTAAGT-3′ | Bcc I | A/G |
| | R: 5′-GATGTCGTCCGCCTCCTC-3′ | | |
| RAB22A | F: 5′-GGGTGCCTGAGTGAGGAAAG-3′ | Taq I | A/T |
| | R: 5′-TTGCATGGATGGAGTCGG-3′ | | |
| PPP1R3D | F: 5′-GGACGTGGAGTTCACCCTGC-3′ | Nae I | A/G |
| | R: 5′-GCGCTAGCAGGAAGGGTGG-3′ | | |

### REFERENCES

All references cited throughout this document are hereby incorporated herein in their entirety by reference.

Chen, A. S., Marsh, D. J., Trumbauer, M. E., Frazier, E. G., Guan, X. M., Yu, H., Rosenblum, C. I., Vongs, A., Feng, Y., Cao, L., Metzger, J. M., Strack, A. M., Camacho, R. E., Mellin, T. N., Nunes, C. N., Min, W., Fisher, J., Gopal-Truter, S., Mactyre, D. E., Chen, H. Y., Van der Ploeg, L. H. T., 2000. Inactivation of the mouse melanocortin-3 receptor results in increased fat mass and reduced lean mass. Nature Genetics, 26, 97-102.
Deussing, J., von Olshausen, I., Peters, C., 2000. Murine and human cathepsin Z: cDNA-cloning, characterization of the genes and chromosomal localization. Biochimica et Biophysica Acta, 1491, 93-106.
Malek, M., J. C. M. Dekkers, H. K. Lee, T. J. Baas, and M. F. Rothschild. 2001. A molecular genome scan analysis to identify chromosomal regions influencing economic traits in the pig. I. Growth and body composition. Mammal. Gen. 12:637-645.
Malek, M., J. C. M. Dekkers, H. K. Lee, T. J. Baas, K. Prusa, E. Huff-Lonergan, and M. F. Rothschild. 2001. A molecular genome scan analysis to identify chromosomal regions influencing economic traits in the pig. II. Meat and muscle composition. Mammal. Gen. 12:630-636.
Russo, V., Fontanesi, L., Davoli, R., Nanni Costa, L., Cagnazzo, M., Buttazzoni, L., Virgili, R., Yerle, M., 2002. Investigation of candidate genes for meat quality in dry-cured ham production: the porcine cathepsin B (CTSB) and cystatin B (CSTB) genes. Animal Genetics, 33, 123-131.
Santamaria, I., Velasco, G., Pendas, A. M., Fueyo, A., Lopez-Otin, C., 1998. Cathepsin Z, a novel human cysteine proteinase with a short propeptide domain and a unique chromosomal location. Journal of Biological Chemistry, 273(27), 16816-16823.
Schalin-Jantti, C., Valli-Jaakola, K., Oksanen, L., Martelin, E., Laitinen, K., Krusius, T., Mustajoki, P., Heikinheimo, M., Kontula, K., 2003. Melanocortin-3 receptor gene variants in morbid obesity. International Journal of Obesity, 27, 70-74.
Yu, S., Gavrilova, O., Chen, H., Lee, R., Liu, J., Pacak, K., Parlow, A. F., Quon, M. J., Reitman, M. L., Weinstein, L. S., 2000. Paternal versus maternal transmission of a stimulatory G-protein α subunit knockout produces opposite effects on energy metabolism. Journal of Clinical Investigation, 105(5), 615-623.
Yu, S., Castle, A., Chen, M., Lee, R., Takeda, K., Weinstein, L. S., 2001. Increased insulin sensitivity in Gₛα knockout mice. Journal of Biological Chemistry, 276(23), 19994-19998.

What is claimed is:

1. A method of selecting a first pig by marker assisted selection of a quantitative trait locus associated with growth traits said method comprising: determining the presence of a locus in the first pig where the locus is located on chromosome 17 in a region of approximately 70 cM to approximately 104 cM and is genetically linked to a polymorphic marker selecting said first animal comprising the locus and thereby selecting the quantitative trait locus associated with growth traits.

2. The method of claim 1 wherein said marker is a polymorphic restriction site selected from the group consisting og Dde I, Msp I, Nae I, Afl III, Alw NI, Bse RI, Taa I, Mse I, Bst UI, Bcc I, Taq I, and Mnl I.

3. A method of selecting a first pig by marker assisted selection of a quantitative trait locus associated with glycolytic potential and average lactate said method comprising: determining the presence of a locus in the first pig where the locus is located on chromosome 17 in a region of approximately 80 cM to approximately 84 cM and is genetically linked to a polymorphic marker selecting said first animal comprising the locus and thereby selecting the quantitative trait locus associated with glycolytic potential and average lactate.

4. The method of claim 3 wherein said marker is a Nae I restriction site.

5. The method of claim 3 wherein said marker is an AFl III restriction site.

6. A method of selecting a first pig by marker assisted selection of a quantitative trait locus associated with color, lab loin hunter, average drip and/or lab loin minolta said method comprising: determining the presence of a locus in the first pig where the locus is located on chromosome 17 in a region of approximately 83 cM to approximately 88 cM and is genetically linked to a polymorphic marker selecting said first animal comprising the locus and thereby selecting the quantitative trait locus associated with color, lab loin hunter, average drip and/or lab loin minolta.

7. The method of claim 6 wherein said marker an Afl III restriction site.

8. The method of claim 6 wherein said marker is an Alw NI restriction site.

9. The method of claim 6 wherein said marker is a Bse RI restriction site.

10. A method of selecting a first pig by marker assisted selection of a quantitative trait locus associated with length, and/or lumbar backfat said method comprising: determining the presence of a locus in the first pig where the locus is located on chromosome 17 in a region of approximately 83 cM to approximately 85 cM and is genetically linked to a polymorphic marker selecting said first animal comprising the locus and thereby selecting the quantitative trait locus associated with length, and/or lumbar backfat.

11. The method of claim 10 wherein said marker is an Afl III restriction site.

12. The method of claim 10 wherein said marker is an Alw NI restriction site.

13. A method of selecting a first pig by marker assisted selection of a quantitative trait locus associated with lumbar backfat said method comprising: determining the presence of a locus in the first pig where the locus is located on chromosome 17 in a region of approximately 85 cM to approximately 90 cM and is genetically linked to a polymorphic marker selecting said first animal comprising the locus and thereby selecting the quantitative trait locus associated with lumbar backfat.

14. The method of claim 13 wherein said marker is an Alw NI restriction site.

15. The method of claim 13 wherein said marker is a Bse RI restriction site.

16. The method of claim 13 wherein said marker is a Taa I restriction site.

17. A method of selecting a first pig by marker assisted selection of a quantitative trait locus associated with growth and fat traits said method comprising: determining the presence of a locus in the first pig where the locus is located on chromosome 17 in a region of approximately 88 cM to approximately 91 cM and is genetically linked to a polymorphic marker selecting said first animal comprising the locus and thereby selecting the quantitative trait locus associated with growth and fat traits.

18. The method of claim 17 wherein said marker is a Mnl I restriction site.

19. The method of claim 17 wherein said marker is a Taa I restriction site.

20. A method of selecting a first pig by marker assisted selection of a quantitative trait locus associated with cooking loss said method comprising: determining the presence of a locus in the first pig where the locus is located on chromosome 17 in a region of approximately 97 cM to approximately 100 cM and is genetically linked to a polymorphic marker selecting said first animal comprising the locus and thereby selecting the quantitative trait locus associated with cooking loss.

21. The method of claim 20 wherein said marker is a Mse I restriction site.

22. The method of claim 20 wherein said marker is a Bst UI restriction site.

23. The method of claim 20 wherein said marker is a Bcc I restriction site.

24. The method of claim 20 wherein said marker is a Taq I restriction site.

25. A method of selecting a first pig by marker assisted selection of a quantitative trait locus associated with color, lab loin hunter, lab loin Minolta, average drip, and/or tenderness said method comprising: determining the presence of a locus in the first pig where the locus is located on chromosome 17 in a region of approximately 100 cM to approximately 104 cM and is genetically linked to a polymorphic marker selecting, said first animal comprising the locus and thereby selecting the quantitative trait locus associated with color, lab loin hunter, lab loin Minolta, average drip, and/or tenderness.

26. The method of claim 25 wherein said marker is a Taq I restriction site.

27. The method of claim 25 wherein said marker is a Bbs I restriction site.

28. The method of claim 25 wherein said marker is an Alw NI restriction site.

29. A method of selecting a first pig by marker assisted selection of a quantitative trait locus associated with growth traits said method comprising: determining the presence of a locus in the first pig where the locus is located on chromosome 17 in a region of approximately 103 cM to approximately 107 cM and is genetically linked to a polymorphic marker selecting said first animal comprising the locus and thereby selecting the quantitative trait locus associated with growth traits.

30. The method of claim 29 wherein said marker is a Nae I restriction site.

31. The method of claim 29 wherein said marker is an AlwNI restriction site.

32. A method of identifying an allele that is associated with growth traits comprising: obtaining a tissue or body fluid sample from an animal; amplifying DNA present in said sample comprising a region of chromosome 17 at a region of approximately 70 to approximately 107 cM detecting the presence of a polymorphic marker in said chromosomal region wherein said marker is associated with phenotypic variation in growth traits.

33. A method of determining a genetic marker which may be used to identify and select animals based upon their growth traits comprising: obtaining a sample of tissue or body fluid from said animals, said sample comprising DNA; amplifying DNA present in said sample in a region of chromosome 17 of approximately 70 to approximately 107 cM, present in said sample from a first animal; determining the presence of a polymorphic allele present in said sample by comparison of said sample with a reference sample or sequence; correlating variability for growth, fatness or meat quality in said animals with said polymorphic allele; so that said allele may be used as a genetic marker for the same in a given group, population, or species.

34. A method of determining a genetic marker which may be used to identify and select animals based upon their meat quality or growth traits comprising: determining a polymorphic allele in useful linkage disequilibrium with the marker disclosed in claim 33.

35. A method of determining a genetic marker which may be used to identify and select animals based upon their meat quality, fatness or growth traits comprising: obtaining a sample of tissue or body fluid from said animals, said sample comprising DNA; amplifying DNA present in said sample in a region of chromosome 17 of approximately 70 to approximately 90 or approximately 97- approximately 107.5 cM, present in said sample from a first animal; determining the presence of a polymorphic allele present in said sample by comparison of said sample with a reference sample or sequence; correlating variability for growth, fatness or meat quality in said animals with said polymorphic allele; so that said allele may be used as a genetic marker for the same in a given group, population, or species.

36. A method of determining a genetic marker which may be used to identify and select animals based upon their meat quality or growth traits comprising: determining a polymorphic allele in useful linkage disequilibrium with the marker disclosed in claim 35.

37. The method of claim 35 wherein said step of determining is selected from the group consisting of: restriction fragment length polymorphism (RFLP) analysis, minisequencing, MALD-TOF, SINE, heteroduplex analysis, single strand conformational polymorphism (SSCP), denaturing gradient gel electrophoresis (DGGE) and temperature gradient gel electrophoresis (TGGE).

38. The method of claim 35 wherein said animal is a pig.

39. The method of claim 35 wherein said amplification includes the steps of: selecting a forward and a reverse primer capable of amplifying a said region of chromosome 17.

40. A method of selecting a first pig by marker assisted selection of a quantitative trait locus associated with average daily gain said method comprising: determining the presence of a locus in the first pig where the locus is located on chromosome 17 in a region of approximately 70 cM to approximately 72 cM and is genetically linked to a polymorphic marker selecting said first animal comprising the locus and thereby selecting the quantitative trait locus associated with average daily gain.

41. The method of claim 40 wherein said marker is a Dde I restriction site.

42. The method of claim 40 wherein said marker is a Msp I restriction site.

43. A method of selecting a first pig by marker assisted selection of a quantitative trait locus associated with marbling score said method comprising: determining the presence of a locus in the first pig where the locus is located on chromosome 17 in a region of approximately 72 cM to approximately 80 cM and is genetically linked to a polymorphic marker selecting said first animal comprising the locus and thereby selecting the quantitative trait locus associated with marbling score.

44. The method of claim 43 wherein said marker is a Msp I restriction site.

45. The method of claim 43 wherein said marker is a Nae I restriction site.

46. A method of selecting a first pig by marker assisted selection of a quantitative trait locus associated with average backfat said method comprising: determining the presence of a locus in the first pig where the locus is located on chromosome 17 in a region of approximately 85 cM to approximately 90 cM and is genetically linked to a polymorphic marker selecting said first animal comprising-the locus and thereby selecting the quantitative trait locus associated with backfat.

47. The method of claim 46 wherein said marker is a Alw NI restriction site.

48. The method of claim 46 wherein said marker is a Bse RI restriction site.

49. The method of claim 46 wherein said marker is a Taa I restriction site.

50. A method of selecting a first pig by marker assisted selection of a quantitative trait locus associated with average backfat, ham hunter, and/or ham minolta said method comprising: determining the presence of a locus in the first pig where the locus is located on chromosome 17 in a region of approximately 97 cM to approximately 99 cM and is genetically linked to a polymorphic marker selecting said first animal comprising the locus and thereby selecting the quantitative trait locus associated with average backfat, ham hunter, and/or ham minolta.

51. The method of claim 50 wherein said marker is a Mse I restriction site.

52. The method of claim 50 wherein said marker is a Bst UI restriction site.

53. A method of selecting a first pig by marker assisted selection of a quantitative trait locus associated with Aloca backfat thickness p2 position said method comprising: determining the presence of a locus in the first pig where the locus is located on chromosome 17 in a region of approximately 100 cM to approximately 103 cM and is genetically linked to a polymorphic marker selecting said first animal comprising the locus and thereby selecting the quantitative trait locus associated with Aloca backfat thickness p2 position.

54. The method of claim 53 wherein said marker is a Alw NI restriction site.

55. The method of claim 53 wherein said marker is a Taq I restriction site.

56. The method of claim 53 wherein said marker is a Bbs I restriction site.
